SEQUENCE LISTING (20 sequences)

SEQ ID NO: 1 - DNA, 58 bp, Mus musculus
cucauggugggcggcguugucauagcaaccgugauugucaucacccuggugauguuga

SEQ ID NO: 2 - DNA, 38 bp, Artificial sequence
  mAPPEx17_39, _62, and _67 oligonucleotides
cagggtgatgacaaucacgguugccaugacaacgccgc

SEQ ID NO: 3 - DNA, 19 bp, Artificial sequence
  mAPPEx17_135A, _139A, _140A, and _141A oligonucleotides
cagggtgatgacaaucacg

SEQ ID NO: 4 - DNA, 19 bp, Artificial sequence
  mAPPEx17_135B, _136B, _139B, _140B, and_141B oligonucleotides
guugccaugacaacgccgc

SEQ ID NO: 5 - DNA, 17 bp, Artificial sequence
  mAPPEx17_144A and _145A oligonucleotides
cagggtgatgacaauca

SEQ ID NO: 6 - DNA, 21 bp, Artificial sequence
  mAPPEx17_144B and _145B oligonucleotides
cgguugccaugacaacgccgc

SEQ ID NO: 7 - DNA, 18 bp, Artificial sequence
  mAPPEx17_136A and _137A oligonucleotides
cagggtgatgacaaucac

SEQ ID NO: 8 - DNA, 18 bp, Artificial sequence
  mAPPEx17_137B and _138B oligonucleotides
uugccaugacaacgccgc

SEQ ID NO: 9 - DNA, 17 bp, Artificial sequence
  mAPPEx17_138A oligonucleotide
cagggtgatgacaauca

SEQ ID NO: 10 - DNA, 20 bp, Artificial sequence
  Forward primer
ctcgacgcaagccataacac

SEQ ID NO: 11 - DNA, 20 bp, Artificial sequence
  Reverse primer
tggaccgactggaaacgtag

SEQ ID NO: 12 - DNA, 23 bp, Artificial sequence
  Forward primer
aactggtgttctttgctgaagat

SEQ ID NO: 13 - DNA, 24 bp, Artificial sequence
  Reverse primer
gcatgatggatggatgtgtactgt

SEQ ID NO: 14 - DNA, 15 bp, Artificial sequence
  Sequencing primer
tcggactcatggtgg

SEQ ID NO: 15 - DNA, 55 bp, Mus musculus
gcggcgttgtcatrgcaaccgtgattgtcatcaccctggtgatgttgaagaagaa

SEQ ID NO: 16 - DNA, 31 bp, Artificial sequence
  Dispensation order
tgcgcgtgtcactagcacgtgatgtcatcac

SEQ ID NO: 17 - DNA, 21 bp, Artificial sequence
  Forward primer
caacatcaccagggtgatgac

SEQ ID NO: 18 - DNA, 20 bp, Artificial sequence
  Reverse primer
catcatcggactcatggtgg

SEQ ID NO: 19 - DNA, 18 bp, Artificial sequence
  Probe
cgttgtcatagcaaccgt

SEQ ID NO: 20 - DNA, 17 bp, Artificial sequence
  Mutant probe
cgttgtcatggcaaccg

(19) United States
(12) Patent Application Publication
(10) Pub. No.: US 2023/0323346 A1
(43) Pub. Date: Oct. 12, 2023

(54) ANTISENSE OLIGONUCLEOTIDES FOR RNA EDITING

(71) Applicant: ProQR Therapeutics II B.V., Leiden (NL)

(72) Inventors: Lenka VAN SINT FIET, Leiden (NL); Alicia SOLER CANTÓN, Leiden (NL)

(21) Appl. No.: 18/006,134

(22) PCT Filed: Jul. 22, 2021
(86) PCT No.: PCT/EP2021/070535

§ 371 Date: Jan. 19, 2023

(30) Foreign Application Priority Data
Jul. 23, 2020 (GB) 2011428.6

(51) Int. Cl. C12N 15/113; C12N 15/10

(52) U.S. Cl. CPC C12N 15/113; C12N 15/102; C12N 2310/11; C12N 2310/346

(57) ABSTRACT

The invention relates to a composition comprising a set of two single stranded antisense oligonucleotides (AONs), wherein one AON is the ‘Editing AON’ and the other AON is the ‘Helper AON’, for use in the deamination of a target adenosine in a target RNA to an inosine, wherein the Editing AON is complementary to a stretch of nucleotides in the target RNA that includes the target adenosine, wherein the Helper AON is complementary to a stretch of nucleotides in the target RNA that is separate from the stretch of nucleotides that is complementary to the Editing AON, wherein the Helper AON has a length of 16 to 22 nucleotides and the Editing AON has a length of 16 to 22 nucleotides.

### TECHNICAL FIELD

The invention relates to the field of medicine, especially to the field of RNA editing, whereby an RNA molecule in a cell is targeted by an antisense oligonucleotide (AON) to specifically change a target nucleotide present in the target RNA molecule. The invention is aimed at amending a specific nucleotide, such as a mutated nucleotide that may cause disease, in the target RNA molecule by engaging an (endogenous) enzyme having deaminase activity.

### BACKGROUND

RNA editing is a natural process through which eukaryotic cells alter the sequence of their RNA molecules, often in a site-specific and precise way, thereby increasing the repertoire of genome encoded RNAs by several orders of magnitude. RNA editing enzymes have been described for eukaryotic species throughout the animal and plant kingdoms, and these processes play an important role in managing cellular homeostasis in metazoans from the simplest life forms (such as Caenorhabditis elegans) to humans. Examples of RNA editing are adenosine (A) to inosine (I) conversions and cytidine (C) to uridine (U) conversions, which occur through enzymes called adenosine deaminase and cytidine deaminase, respectively. The most extensively studied RNA editing system is the system involving the adenosine deaminase enzyme. The adenosine deaminases are part of a family of enzymes known as Adenosine Deaminases acting on RNA (ADAR), which include human deaminases hADAR1 and hADAR2, as well as hADAR3. However, for hADAR3 no deaminase activity has been shown yet.

ADAR is a multi-domain protein, comprising a catalytic domain, and two to three double-stranded RNA recognition domains, depending on the enzyme in question. Each recognition domain recognizes a specific double stranded RNA (dsRNA) sequence and/or conformation. The catalytic domain does also play a role in recognizing and binding a part of the dsRNA helix, although the key function of the catalytic domain is to convert an A into I in a nearby, more or less predefined, position in the target RNA, by deamination of the nucleobase. Inosine is read as guanosine by the translational machinery of the cell, meaning that, if an edited adenosine is in a coding region of an mRNA or pre-mRNA, it can recode the protein sequence. A>I conversions may also occur in 5′ non-coding sequences of a target mRNA, creating new translational start sites upstream of the original start site, which gives rise to N-terminally extended proteins, or in the 3′ UTR or other non-coding parts of the transcript, which may affect the processing and/or stability of the RNA. In addition, A>I conversions may take place in splice elements in introns or exons in pre-mRNAs, thereby altering the pattern of splicing. As a result, exons may be (partly or completely) included or skipped.

The use of oligonucleotides to edit a target RNA applying adenosine deaminase has been described (e.g. Montiel-Gonzalez et al. 2013. Proc Natl Acad Sci USA 110(45): 18285-18290; Vogel et al. 2014. Angewandte Chemie Int Ed 53: 267-271; Woolf et al. 1995. Proc Natl Acad Sci USA 92: 8298-8302). A disadvantage of the method described by Montiel-Gonzalez et al. (2013) is the need fora fusion protein consisting of the boxB recognition domain of bacteriophage lambda N-protein, genetically fused to the adenosine deaminase domain of a truncated natural ADAR protein. It requires target cells to be either transduced with the fusion protein, which is a major hurdle, or that target cells are transfected with a nucleic acid construct encoding the engineered adenosine deaminase fusion protein for expression. The system described by Vogel et al. (2014) suffers from similar drawbacks, in that it is not clear how to apply the system without having to genetically modify the ADAR first and subsequently transfect or transform the cells harboring the target RNA, to provide the cells with this genetically engineered protein. Clearly, these systems are not readily adaptable for use in humans (e.g. in a therapeutic setting). The oligonucleotides of Woolf et al. (1995) that were 100% complementary to the target RNA sequences, only appeared to function in cell extracts or in Xenopus oocytes by microinjection, and suffered from severe lack of specificity: nearly all adenosines in the target RNA strand that was complementary to the antisense oligonucleotide were edited. An oligonucleotide, 34 nucleotides in length, wherein each nucleotide carried a 2′-O-methyl (2′-OMe) modification, was tested and shown to be inactive in Woolf et al. (1995). In order to provide stability against nucleases, a 34-mer RNA, modified with 2′-OMe and modified with phosphorothioate (PS) linkages at the 5′- and 3′- terminal 5 nucleotides, was also tested. It was shown that the central unmodified region of this oligonucleotide could promote editing of the target RNA by endogenous ADAR, with the terminal modifications providing protection against exonuclease degradation. However, this system did not show deamination of a specific target adenosine in the target RNA sequence. As mentioned, nearly all adenosines opposite to an unmodified nucleotide in the antisense oligonucleotide were edited (therefore nearly all adenosines opposite nucleotides in the central unmodified region, if the 5′- and 3′-terminal 5 nucleotides of the antisense oligonucleotide were modified, or nearly all adenosines in the target RNA strand if no nucleotides were modified).

It is known in the art that ADAR may act on any dsRNA. Through a process sometimes referred to as ‘promiscuous editing’, the enzyme will edit multiple A's in the dsRNA. Hence, there is a need for methods and means that circumvent such promiscuous editing and only target specific adenosines in a target RNA molecule to become therapeutic applicable. Vogel et al. (2014) showed that such off-target editing can be suppressed by using 2′-OMe-modified nucleotides in the oligonucleotide at positions opposite to adenosines that should not be edited, and used a non-modified nucleotide directly opposite to the specifically targeted adenosine on the target RNA. However, the specific editing effect at the target nucleotide has not been shown to take place without the use of recombinant ADAR enzymes having covalent bonds with the AON.

WO 2016/097212 discloses antisense oligonucleotides (AONs) for the targeted editing of RNA, wherein the AONs are characterized by a sequence that is complementary to a target RNA sequence (therein referred to as the ‘targeting portion’) and by the presence of a stem-loop structure (therein referred to as the ‘recruitment portion’), which is preferably non-complementary to the target RNA. Such oligonucleotides are referred to as ‘self-looping AONs’. The recruitment portion acts in recruiting a natural ADAR enzyme present in the cell to the dsRNA formed by hybridization of the target sequence with the targeting portion. Due to the recruitment portion there is no need for conjugated entities or presence of modified recombinant ADAR enzymes.

The recruitment portion was a stem-loop structure mimicking either a natural substrate (e.g. the GluB receptor) or a Z-DNA structure known to be recognized by the dsRNA binding regions of ADAR enzymes. A stem-loop structure can be an intermolecular stem-loop structure, formed by two separate nucleic acid strands, or an intramolecular stem loop structure, formed within a single nucleic acid strand. The stem-loop structure of the recruitment portion as described in WO 2016/097212 is intramolecular, formed within the AON itself, and able to attract ADAR. WO 2017/220751 and WO 2018/041973 describe AONs that do not comprise a recruitment portion but that are (almost fully) complementary to the targeted area, except for one or more mismatches, or so-called ‘wobbles’ or bulges. The sole mismatch may be the nucleotide opposite the target adenosine, but in other embodiments AONs were described with multiple bulges and/or wobbles when attached to the target sequence area. It appeared possible to achieve in vitro, ex vivo and in vivo RNA editing with AONs lacking a recruitment portion and with endogenous ADAR enzymes when the sequence of the AON was carefully selected such that it could attract ADAR. The ‘orphan nucleotide’, which is defined as the nucleotide in the AON that is positioned directly opposite the target adenosine in the target RNA molecule, did not carry a 2′-OMe modification.

The orphan nucleotide could also be a DNA nucleotide (carrying no 2′ modification in the sugar entity), wherein the remainder of the AON did carry 2′-O-alkyl modifications at the sugar entity (such as 2′-OMe), or the nucleotides within the so-called ‘Central Triplet’ (=the orphan nucleotide with its two direct neighbouring nucleotides within the AON) or directly surrounding the Central Triplet contained particular chemical modifications (or were DNA) that further improved the RNA editing efficiency and/or increased the resistance against nucleases. Such effects could even be further improved by using sense oligonucleotides (SONs) that ‘protected’ the AONs against breakdown, which was described in WO2018/134301.

WO 2019/158475, WO 2019/219581, PCT/EP2020/053283 (unpublished) and PCT/EP2020/059369 (unpublished) describe AONs for RNA editing that have particular chemical modifications in the oligonucleotide backbone and/or sugar moiety at very specific positions to increase the stability of the AONs and/or the efficiency of RNA editing, whereas PCT/EP2020/060291 (unpublished) describes the use of AONs to inhibit RNA editing at specified positions, for instance where the (naturally occurring) RNA editing results in disease, such as seen with certain cancers and viral infections.

Despite the wide variety of efforts outlined above, it appears that RNA editing by using antisense oligonucleotides was mainly achieved with relatively large molecules. The problem is that the larger the AON, the more difficult it becomes to have it enter the cell on its own. Moreover, a long AON is more prone to breakdown than a short AON. Hence, there remains a need for improved, and in this particular case, shorter compounds that can still utilise (endogenous) cellular pathways and enzymes that have deaminase activity, such as naturally expressed ADAR enzymes to more specifically and more efficiently edit endogenous nucleic acids in mammalian cells, even in whole organisms, to alleviate disease.

### SUMMARY OF THE INVENTION

The invention relates to a composition comprising a set of two single stranded antisense oligonucleotides (AONs), wherein one AON is the ‘Editing AON’ and the other AON is the ‘Helper AON’, for use in the deamination of a target adenosine in a target RNA, wherein the Editing AON is complementary to a stretch of nucleotides in the target RNA that includes the target adenosine, wherein the nucleotide in the Editing AON that is directly opposite the target adenosine is the ‘orphan nucleotide’, which is a cytidine that is not modified with 2′-OMe or 2′-MOE, wherein the Helper AON is complementary to a stretch of nucleotides in the target RNA that is separate from the stretch of nucleotides that is complementary to the Editing AON, wherein the Helper AON has a length of 16 to 22 nucleotides and the Editing AON has a length of 16 to 22 nucleotides. In a preferred aspect, the Helper AON and Editing AON form the double stranded complex with the target RNA in a consecutive manner, wherein the Helper AON is complementary to a stretch of nucleotides in the target RNA that is located at the 3′ side of the stretch of nucleotides in the target RNA that is complementary to the Editing AON, and wherein there is no nucleotide gap between sequences complementary to the Helper AON and the Editing AON. In a preferred aspect, the set, after forming the double stranded complex with the target RNA, is configured to recruit an endogenous ADAR enzyme to bring about the deamination of the target adenosine into an inosine. In a preferred aspect, the Editing AON is 19, 20, or 21 nucleotides in length and the Helper AON is 17, 18, or 19 nucleotides in length. As outlined herein, particular good results were obtained when the Editing AON is 21 nucleotides in length and the Helper AON is 17 nucleotides in length, together placed on the target RNA molecule as a consecutive stretch of 38 nucleotides, without a gap between the Helper and Editing AONs. In another preferred aspect, the orphan nucleotide in the Editing AON is the 6^{th}, 7^{th} or 8^{th} nucleotide from the 5′ end.

In another embodiment, the invention relates to a method for the deamination of at least one target adenosine in a target RNA in a cell, the method comprising the steps of: providing the cell with a set of AONs comprising a Helper AON and an Editing AON present in a composition according to the invention; allowing annealing of the AONs to the target RNA to form a double stranded nucleic acid molecule; allowing an ADAR enzyme endogenously present in said cell to complex with the double stranded nucleic acid molecule and to deaminate the target adenosine in the target RNA to an inosine; and optionally identifying the presence of the deaminated nucleotide in the target RNA.

The invention also relates to an in vitro, in vivo, or ex vivo method for the deamination of at least one target adenosine, present in a target RNA, the method comprising the steps of: providing a composition comprising a set of two AONs according to the invention; allowing annealing of the AONs to the target RNA to form a double stranded nucleic acid molecule; allowing an ADAR enzyme to complex with the double stranded nucleic acid molecule and to deaminate the target adenosine in the target RNA to an inosine; and identifying the presence of the deaminated adenosine in the target RNA.

The invention also relates to a single stranded AON, referred to as an ‘Editing AON’, wherein the AON is capable of forming a double stranded complex with a target RNA in a cell, for use in the deamination of a target nucleotide in the target RNA, wherein the Editing AON is complementary to a stretch of nucleotides in the target RNA that includes the target nucleotide, wherein the nucleotide in the Editing AON that is directly opposite the target adenosine is the ‘orphan nucleotide’ that is not modified with 2′-OMe or 2′-MOE, characterized in that the Editing AON has a length of 16 to 22 nucleotides, preferably 19, 20, or 21 nucleotides, more preferably 21 nucleotides. The invention provides a 16 to 22 nucleotide long Editing AON comprising a sequence configured for the deamination of preferably an adenosine as the target nucleotide. Despite its short structure, the Editing AON of the invention can engage an enzyme with deamination activity, even in the absence of a Helper AON as disclosed herein, and wherein the target nucleotide is an adenosine that is deaminated by the deaminating enzyme to an inosine. In a preferred aspect, the Editing AON of the present invention comprises at least one nucleotide comprising a 2′-OMe or a 2′-MOE ribose modification, and the orphan nucleotide does not carry a 2′-OMe or a 2′-MOE ribose modification.

### BRIEF DESCRIPTION OF THE DRAWINGS

One or more embodiments of the invention will now be described, by way of example only, with reference to the accompanying drawings, in which:

FIG. 1 shows (A) the target mouse Amyloid Precursor Protein (mAPP) RNA sequence (SEQ ID NO:1) from 5′ to 3′ and below it the sequences of 21 antisense oligonucleotides as indicated. AONs mAPPEx17_39, _62, and _67 have the sequence of SEQ ID NO:2. AONs mAPPEx17_135A, _139A, _140A, and _141A have the sequence of SEQ ID NO:3. AONs mAPPEx17_135B, _136B, _139B, _140B, and _141B have the sequence of SEQ ID NO:4. AONs mAPPEx17_144A and _145A have the sequence of SEQ ID NO:5. AONs mAPPEx17_144B and _145B have the sequence of SEQ ID NO:6. AONs mAPPEx17_136A and mAPPEx17_137A have the sequence of SEQ ID NO:7. AONs mAPPEx17_137B and mAPPEx17_138B have the sequence of SEQ ID NO:8. AON mAPPEx17_138A has the sequence of SEQ ID NO:9. The chemical modifications in each of the AONs are provided in (B): an asterisk indicates a phosphorothioate (PS) linkage between two nucleosides; not underlined lower case nucleotides are modified with 2′-OMe; underlined lower case nucleotides are modified with 2′-MOE; upper case nucleotides are DNA; and the bold upper case C represents the orphan nucleotide (DNA) directly opposite the adenosine in the target sequence that needs to be edited.

FIG. 2 shows the editing percentage in an in vitro biochemical assay, achieved by using the 38 nt long mAPPEx17-62 as a single AON and as a positive control for RNA editing, in comparison to the levels achieved with: i) a set of split AONs comprising the 19 nt long mAPPEx17-139A as the Helper AON and the 19 nt long mAPPEx17-139B as the Editing AON; and with ii) a set of split AONs comprising the 17 nt long mAPPEx17-144A as the Helper AON and the 21 nt long mAPPEx17-144B as the Editing AON. It is clearly noted that both sets were capable of providing proper, quick and high levels of RNA editing, wherein the 17/21 nt set (mAPPEx17-144A and -B) performed similarly to the 38 nt positive control AON (mAPPEx17_62), and slightly performed more efficient than the 19/19 nt set (mAPPEx17-139A and -B).

FIG. 3 shows the editing percentage in an in vitro biochemical assay, achieved by using the 38 nt long mAPPEx17-67 as a single AON and as a positive control for RNA editing, in comparison to the levels achieved with: i) a set of split AONs comprising the 19 nt long mAPPEx17_140A as the Helper AON and the 19 nt long mAPPEx17_140B as the Editing AON;

with ii) a set of split AONs comprising the 19 nt long mAPPEx17_141A as the Helper AON and the 19 nt long mAPPEx17_141B as the Editing AON; and iii) a set of split AONs comprising the 17 nt long mAPPEx17_145A as the Helper AON and the 21 nt long mAPPEx17_145B as the Editing AON. All three sets were able to provide significant RNA editing, with the set of mAPPEx17_145A and -B outperforming the two other sets.

FIG. 4 shows the editing percentage in an in vitro biochemical assay, achieved by using the 38 nt long mAPPEx17_62 as a single AON and as a positive control for RNA editing, in comparison to the levels achieved with: i) a set of split AONs comprising the 19 nt long mAPPEx17-139A as the Helper AON and the 19 nt long mAPPEx17-139B as the Editing AON; ii) mAPPEx17_139B as a single Editing AON; iii) a set of split AONs comprising the 17 nt long mAPPEx17-144A as the Helper AON and the 21 nt long mAPPEx17-144B as the Editing AON; and iv) mAPPEx17_144B as a single Editing AON. Whereas both sets resulted in efficient RNA editing, also the Editing AON mAPPEx17_144B gave a high level of RNA editing when used alone. In contrast, the use of mAPPEx17_139B as an Editing AON when used alone, did not give high levels of editing.

FIG. 5 shows the editing percentage in an in vitro biochemical assay, achieved by using the 38 nt long mAPPEx17_67 as a single AON and as a positive control for RNA editing, in comparison to the levels achieved with: i) a set of split AONs comprising the 19 nt long mAPPEx17_141A as the Helper AON and the 19 nt long mAPPEx17_141B as the Editing AON; ii) mAPPEx17_141B as a single Editing AON; iii) a set of split AONs comprising the 17 nt long mAPPEx17_145A as the Helper AON and the 21 nt long mAPPEx17_145B as the Editing AON; and iv) mAPPEx17_145B as a single Editing AON. As shown in FIG. 3, the set of mAPPEx17_141A and -B did give a moderate level of RNA editing and the use of the single mAPPEx17)141B as the Editing AON did not yield a high level of editing. The level achieved with the set of mAPPEx17_145A and -B was comparable to what was shown in FIG. 3, and the use of the single mAPPEx17_145B as the Editing AON gave a proper level of RNA editing.

FIG. 6 shows the editing percentage obtained after transfection of a variety of sets of Editing and Helper AONs, as well as separate Editing AONs without the co-transfection of a Helper AON in cells, as indicated, wherein the sets of AONs and the single Editing AONs can recruit endogenously present ADAR in the cell to bring about deamination of a target adenosine. Negative controls were a non-transfected sample (NT), a mock transfected sample and a transfection with a control scrambled AON. While two positive control AONs, mAPPEx17_62 and _67 (each 38 nt long) were able to reach ˜13% to ˜28% editing, the sets as well as the single AONs were also able to bring about RNA editing, with the sets mAPPEx17_140A/B, mAPPEx17_141A/B, mAPPEx17_145A/B and the single Editing AON mAPPEx17_141B performing best.

FIG. 7 shows the editing percentage in an in vitro biochemical assay, achieved by using the 38 nt long mAPPEx17_39 as a single AON and as a positive control for RNA editing, in comparison to the levels achieved with: i) a set of split AONs comprising the 19 nt long mAPPEx17-135A as the Helper AON and the 19 nt long mAPPEx17-135B as the Editing AON; ii) a set of split and 1 nt gapped AONs comprising the 18 nt long mAPPEx17-136A as the Helper AON and the 19 nt long mAPPEx17-136B as the Editing AON; iii) a set of split and 2 nt gapped AONs comprising the 18 nt long mAPPEx17-137A as the Helper AON and the 18 nt long mAPPEx17-137B as the Editing AON; and a set of split and 3 nt gapped AONs comprising the 17 nt long mAPPEx17-138A as the Helper AON and the 18 nt long mAPPEx17-138B as the Editing AON. It appears that the split AONs do not form together a consecutive sequence of complementarity (in other words, a ‘gap’ exists when bound to the target RNA) RNA editing can still be achieved, but is less efficient than when the split AONs have two sequences that are consecutive to each other and no gap is formed: see the set of mAPPEx17_135A and -B that performs as well as the single 38 nt long mAPPEx17_39 AON.

### DETAILED DESCRIPTION

All previously described RNA editing antisense oligonucleotides (AONs, herein and elsewhere often also referred to as ‘editing oligonucleotides’, and often abbreviated to ‘EONs’) consist of a single stretch of nucleotides in a single molecule with a sequence that is either fully or partially complementary to the target RNA sequence. Even though the wish is to have an AON with a relatively short length, thus far the most optimal and tested AONs have a length between approximately 35 and 60 nucleotides. This is, in general, much longer than typical AONs that are used for exon skipping, splice modulation, or transcription/translation inhibition. Such AONs generally have an optimal length of 16 to 24 nucleotides. A common limiting factor in AON-based therapies are the oligonucleotide's ability to be taken up by the cell (when delivered per se, or ‘naked’ without applying a delivery vehicle), its biodistribution and its resistance to nuclease-mediated breakdown. The skilled person is aware, and it has been described in detail in the art, that a variety of chemical modifications can assist in overcoming such limitations. Examples of such now commonly used chemical modifications are the 2′-O-methyl (2′-OMe) and 2′-O-methoxyethyl (2′-MOE) modifications of the sugar and the use of phosphorothioated (PS) linkages between nucleosides. PCT/EP2020/059369 (unpublished) describes the use of methylphosphonate (MP) linkage modifications at certain positions surrounding the orphan nucleotide in the AON. Also, it was found that phosphonoacetate linkage modifications and/or unlocked nucleic acid (UNA) ribose modifications of some, but not all, positions in the AON appeared compatible with efficient engagement of an enzyme with nucleotide deamination activity and with subsequent deamination (PCT/EP2020/053283, unpublished). Whereas the properties of phosphonoacetate and UNA modifications were known as such, the compatibility thereof with engagement of enzymes with nucleotide deamination activity and with the deamination reaction was not known. In a UNA modification, there is no carbon-carbon bond between the ribose 2′ and 3′ carbon atoms. UNA ribose modifications therefore increase the local flexibility in oligonucleotides. UNAs can lead to effects such as improved pharmacokinetic properties through improved resistance to degradation. UNAs can also decrease toxicity and may participate in reducing off-target effects. A UNA ribose modification should preferably be avoided at the orphan nucleotide as disruption of binding with the enzyme with nucleotide deaminase activity would be significant. The UNA ribose modification may be the only ribose modification in the AON, but the UNA modification may exist in addition to modifications to the ribose 2′ group, either at positions different to the UNA modifications or at the same positions as the UNA modifications. The ribose 2′ groups in the AON can be independently selected from 2′-H (i.e. DNA), 2′-OH (i.e. RNA), 2′-OMe, 2′-MOE, 2′-F, or 2′-4′-linked (for instance a locked nucleic acid (LNA)), or other 2′ substitutions. The 2′-4′ linkage can be selected from linkers known in the art, such as a methylene linker or constrained ethyl linker (cEt). Nonetheless, because the thus-far used EONs are relatively long, biodistribution, cell-entry and efficiency in RNA editing in vivo remain a concern and may be limited. The inventors of the present invention have now found solutions to these problems, as outlined herein. They contemplated using a set of AONs (preferably with only two oligonucleotides in a set) and subsequently they used only a single, short AON while still achieving efficient RNA editing.

The invention relates to a composition comprising a set of two single stranded antisense oligonucleotides (AONs), wherein one AON is the ‘Editing AON’ and the other AON is the ‘Helper AON’, for use in the deamination of a target nucleotide (preferably adenosine) in a target RNA, wherein the Editing AON is complementary to a stretch of nucleotides in the target RNA that includes the target adenosine, wherein the nucleotide in the Editing AON that is directly opposite the target nucleotide is the ‘orphan nucleotide’ (when the target nucleotide is an adenosine, the orphan nucleotide is preferably a cytidine), that is not modified with 2′-OMe or 2′-MOE, wherein the Helper AON is complementary to a stretch of nucleotides in the target RNA that is separate from the stretch of nucleotides that is complementary to the Editing AON, wherein the Helper AON has a length of 16 to 22 nucleotides and the Editing AON has a length of 16 to 22 nucleotides. In a preferred aspect, the Helper AON and Editing AON form the double stranded complex with the target RNA in a consecutive manner, wherein the Helper AON is complementary to a stretch of nucleotides in the target RNA that is located at the 3′ side of the stretch of nucleotides in the target RNA that is complementary to the Editing AON, and wherein there is no nucleotide gap between sequences complementary to the Helper AON and the Editing AON. This means that the AONs do not overlap such that they are complementary to the same sequence, or parts thereof, in the target RNA. As shown herein, RNA editing can be achieved when a gap of 1, 2 or 3 nucleotides exist between the Helper AON and the Editing AON, the best results were obtained when the Helper AON and the Editing AON were aligned on the target RNA molecule in a consecutive way, without a gap. Preferably, the Helper AON is 100% complementary to the target RNA. Preferably, the Editing AON, besides when there is a mismatch between the orphan nucleotide and the target nucleotide, is fully complementary to the target RNA. In a preferred aspect, the set, after forming the double stranded complex with the target RNA, can recruit an endogenous ADAR enzyme to bring about the deamination of the target nucleotide.

The nucleotide numbering in the Editing AON is such that the orphan nucleotide is number 0 and the nucleotide 5′ from the orphan nucleotide is number +1. Counting is further positively incremented towards the 5′ end and negatively (−) incremented towards the 3′ end, wherein the first nucleotide 3′ from the orphan nucleotide is number −1.

The internucleoside linkage numbering in the Editing AON is such that linkage number 0 is the linkage 5′ from the orphan nucleotide, and the linkage positions in the oligonucleotide are positively (+) incremented towards the 5′ end and negatively (−) incremented towards the 3′ end.

Preferably, the Editing AON comprises one or more phosphorothioate (PS) linkages. Preferably the PS linkages connect the most terminal 4, 5, 6, 7, or 8 nucleotides on each end of the Editing AON. More preferably, PS linkages are present in the Editing AON at linkage positions +6, +5, +4, +3, +2, and +1, for instance when the Editing AON is 21 nucleotides in length. Also, preferably, PS linkages are present in the Editing AON at linkage positions −7, −8, −9, −10, −11, −12, and −13, for instance when the Editing AON is 21 nucleotides in length.

Preferably, the Editing AON comprises at least one nucleotide with a sugar moiety that comprises a 2′-OMe modification. Preferred positions for the 2′-OMe modification at the 5′ end of the AON in a 21-nt long AON according to the invention are nucleotide position +2, +3, +3, +4, +5, +6 and/or +7 (preferably all nucleotides located 5′ from the +1 position). Preferred positions for the 2′-OMe modification at the 3′ end of the AON in a 21-nt long AON according to the invention are nucleotide position −2, −5, −6, −7, −8, −9, −10, −11, −12 and/or −13. It is preferred to have all nucleotides that are located 3′ from the −4 position to be modified with a 2′-OMe group in the sugar moiety.

Preferably, the Editing AON comprises at least one nucleotide with a sugar moiety that comprises a 2′-MOE modification. Preferred positions for the nucleotides carrying a 2′-MOE modification are positions +1 and/or −4 in the Editing AON.

Preferably, the orphan nucleotide carries a 2′-H in the sugar moiety and is therefore referred to as a DNA nucleotide. The nucleotide 3′ and/or 5′ from the orphan nucleotide are also DNA, more preferably the nucleotide at the 3′ (position −1).

Preferably, the Editing AON comprises at least one methylphosphonate (MP) internucleoside linkage according to the following structure:

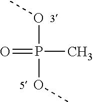

A preferred position for an MP linkage in an Editing AON according to the invention is linkage position −1, thereby connecting the nucleoside at position −1 with the nucleoside at position −2, although other positions for MP linkages are not explicitly excluded.

Preferably, the Editing AON comprises at least one nucleotide with a sugar moiety that comprises a 2′-fluoro (2′-F) modification. A preferred position for the nucleotide that carries a 2′-F modification is position −3 in the Editing AON.

Preferably, the Editing AON comprises at least one phosphonoacetate internucleoside linkage.

Preferably, the Editing AON according to the invention comprises at least one nucleotide comprising an unlocked nucleic acid (UNA) ribose modification.

The preferred modifications outlined above may also be introduced into the Helper AON, although that AON does not suffer from instability issues seen with the Editing AON in that the orphan nucleotide cannot carry a 2′ modification, such as 2′-OMe or 2′-MOE, and should preferably be DNA to function properly. The Helper AON preferably comprises four PS linkage modifications at each end of the AON, thereby connecting the five terminal nucleosides at each end. The Helper AON is, in addition, preferably fully modified with 2′-OMe and/or 2′-MOE modifications, in which the number of nucleotides carrying a 2′-OMe or 2′-MOE modification may differ. DNA is generally not present in the Helper AON, but such is not explicitly excluded. The Helper AON may be 100% complementary to the target RNA, but may also comprise one or more mismatches, wobbles, or bulges, which may add in the capability to recruit the deaminase enzyme.

Preferably, the Editing AON is 19, 20, or 21 nucleotides in length, wherein the orphan nucleotide is the 6^{th}, 7^{th} or 8^{th} nucleotide from the 5′ end, and wherein the Helper AON is 17, 18, or 19 nucleotides in length. The invention further relates to a composition for use according to the invention, further comprising a pharmaceutically acceptable carrier.

The invention relates to a single stranded AON, referred to as an ‘Editing AON’, wherein the AON is capable of forming a double stranded complex with a target RNA in a cell, for use in the deamination of a target nucleotide in the target RNA, wherein the Editing AON is complementary to a stretch of nucleotides in the target RNA that includes the target nucleotide, wherein the nucleotide in the Editing AON that is directly opposite the target adenosine is the ‘orphan nucleotide’ that is not modified with 2′-OMe or 2′-MOE, characterized in that the Editing AON has a length of 16 to 22 nucleotides, preferably 19, 20, or 21 nucleotides, more preferably 21 nucleotides. The invention provides a 16 to 22 nucleotide long Editing AON comprising a sequence configured for the deamination of preferably an adenosine as the target nucleotide. Despite its short structure, the Editing AON of the invention can engage an enzyme with deamination activity, even in the absence of a Helper AON as disclosed herein, and preferably the target nucleotide is an adenosine that is deaminated by the deaminating enzyme to an inosine. In a preferred aspect, the Editing AON of the present invention comprises at least one nucleotide comprising a 2′-OMe or a 2′-MOE ribose modification, and the orphan nucleotide does not carry a 2′-OMe or a 2′-MOE ribose modification.

A composition according to the invention or a single Editing AON according to the invention is preferably used in the treatment or prevention of a genetic disorder, preferably selected from the group consisting of: Cystic fibrosis, Hurler Syndrome, alpha-1-antitrypsin (A1AT) deficiency, Parkinson's disease, Alzheimer's disease, albinism, Amyotrophic lateral sclerosis, Asthma, β-thalassemia, CADASIL, Charcot-Marie-Tooth disease, Chronic Obstructive Pulmonary Disease (COPD), Distal Spinal Muscular Atrophy (DSMA), Duchenne/Becker muscular dystrophy, (Dystrophic) Epidermolysis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber Congenital Amaurosis (such as LCA10), Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anemia, Spinal Muscular Atrophy, Stargardt disease, Tay-Sachs Disease, Usher syndrome (such as Usher syndrome type I, type II, and type III), X-linked immunodeficiency, Sturge-Weber Syndrome, and cancer.

The invention also relates to a method for the deamination of at least one target adenosine in a target RNA in a cell, the method comprising the steps of: providing the cell with a set of AONs comprising a Helper AON and an Editing AON according to the invention or a single Editing AON according to the invention; allowing annealing of the AON(s) to the target RNA to form a double stranded nucleic acid molecule; allowing an ADAR enzyme endogenously present in said cell to complex with the double stranded nucleic acid molecule and to deaminate the target nucleotide (preferably an adenosine) in the target RNA; and optionally identifying the presence of the deaminated nucleotide in the target RNA. The identification step in the method of the invention may comprise: sequencing a region of the target RNA, wherein the region comprises the position of the target nucleotide; assessing the presence of a functional, elongated, full length and/or wild type protein when the target nucleotide is an adenosine in a UGA or UAG stop codon, which is edited to a UGG codon through the deamination; assessing, when the target RNA is pre-mRNA, whether splicing of the pre-mRNA was altered by the deamination; or using a functional read-out, wherein the target RNA after the deamination encodes a functional, full length, elongated and/or wild type protein.

The invention also relates to an in vivo, ex vivo, or in vitro method for the deamination of at least one target adenosine, present in a target RNA, the method comprising the steps of: providing a set of two AONs according to the invention or a single Editing AON according to the invention; allowing annealing of the AON(s) to the target RNA to form a double stranded nucleic acid molecule; allowing an ADAR enzyme to complex with the double stranded nucleic acid molecule and to deaminate a target adenosine in the target RNA to an inosine; and identifying the presence of the deaminated adenosine in the target RNA.

In all aspects of the invention, the enzyme with nucleotide deaminase activity is preferably an ADAR enzyme, more preferably ADAR1 or ADAR2. In a highly preferred aspect, the Editing AON is an RNA editing single-stranded AON that targets a pre-mRNA or an mRNA, wherein the target nucleotide is preferably an adenosine in the target RNA, wherein the adenosine is deaminated to an inosine, which is being read as a guanosine by the translation machinery. In a further preferred embodiment, the adenosine is located in a UGA or UAG stop codon, which is edited to a UGG codon; or wherein two target nucleotides are the two adenosines in a UAA stop codon, which codon is edited to a UGG codon through the deamination of both target adenosines, wherein two nucleotides in the oligonucleotide mismatch with the target nucleic acid.

The Helper AON and the Editing AON according to the invention can comprise internucleoside linkage modifications other than, or in addition to, the PS linker modifications. In one embodiment one such other internucleoside linkage can be a phosphonoacetate or a methylphosphonate modified linkage. In another embodiment, the internucleoside linkage can be a phosphodiester wherein the OH group of the phosphodiester has been replaced by alkyl, alkoxy, aryl, alkylthio, acyl, -NR1R1, alkenyloxy, alkynyloxy, alkenylthio, alkynylthio, -S−Z+, -Se−Z+, or -BH3−Z+, and wherein R1 is independently hydrogen, alkyl, alkenyl, alkynyl, or aryl, and wherein Z+is ammonium ion, alkylammonium ion, heteroaromatic iminium ion, or heterocyclic iminium ion, any of which is primary, secondary, tertiary or quaternary, or Z is a monovalent metal ion. Preferred is the use of PS linkage modifications.

In the Editing AON of the present invention, the orphan nucleotide generally comprises a deoxyribose with a 2′-H group, and preferably does not comprise a ribose carrying a 2′-OMe or a 2′-MOE modification. Further, the Editing AON of the present invention generally further comprises 2′-MOE modifications at other positions within the AON. The same holds true for the Helper AON. The AONs of the present invention preferably do not comprise a recruitment portion as described in WO 2016/097212. The AONs of the present invention do not comprise a portion that can form an intramolecular stem-loop structure. The AONs do not include a 5′-terminal O6-benzylguanine modification. The AONs do not include a 5′-terminal amino modification. The AONs are not covalently linked to a SNAP-tag domain. The internucleotide linkage numbering in an AON according to the present invention is such that linkage number 0 is the linkage 5′ from the orphan nucleotide, which is itself the “0” nucleotide.

The present invention also relates to AONs that may target premature termination stop codons (PTCs) present in the (pre)mRNA to alter the adenosine present in the stop codon to an inosine (read as a G), which in turn then results in read-through during translation and a full length functional protein. The teaching of the present invention, as outlined herein, is applicable for all genetic diseases that may be targeted with AONs and may be treated through RNA editing. One example is Usher syndrome type II caused by mutations in the USH2A gene.

In one aspect, the invention relates to an AON (or a set of two AONs) capable of forming a double stranded complex with a target RNA molecule in a cell, for use in the deamination of a target adenosine in a disease-related splice mutation present in the target RNA molecule, wherein the orphan nucleotide in the Editing AON does not carry a 2′-OMe modification; wherein the nucleotide directly 5′ and/or 3′ from the orphan nucleotide (which nucleotides — together with the orphan nucleotide—form the Central Triplet) carry a sugar modification and/or a base modification to render the AON more stable and/or more effective in RNA editing; and preferably wherein at least one linkage in the AON is modified to comprise a PS modification. In one preferred aspect, at least one internucleoside linkage connecting two nucleosides carries an MP modification. When two nucleotides in the Editing AON are DNA all others may be RNA and may be 2′-OMe or 2′-MOE modified. In one aspect, the AON, or each of the AONs (when in a set of two) according to the invention comprises 2, 3, or 4 mismatches, wobbles and/or bulges with the complementary target RNA region. Preferably, the nucleotide that is opposite the target adenosine is a cytidine, a deoxycytidine, a uridine, a deoxyuridine, or is a basic. PCT/US2020/037580 (unpublished) describes the application of cytidine analogs at the orphan nucleotide position to render the AON more efficient in RNA editing because such cytidine analogs interact with the ADAR enzyme in a firmer fashion. The incorporation of a cytidine analog (such as pseudoisocytidine (piC) or Benner's base Z (dZ)) at the orphan nucleotide position is also a preferred aspect of the present invention. When the nucleotide opposite the target adenosine is a cytidine or a deoxycytidine, the AON comprises at least one mismatch with the target RNA molecule. When the nucleotide opposite the target adenosine is a uridine or a deoxyuridine, the AON may be 100% complementary and not have any mismatches, wobbles, or bulges in relation to the target RNA. However, in one aspect one or more additional mismatches, wobbles and/or bulges may be present between AON and target RNA whether the nucleotide opposite the target adenosine is a cytidine, a deoxycytidine, a pseudoisocytidine, a Benner's base Z, a uridine, or a deoxyuridine. In another preferred embodiment, the nucleotide directly 5′ and/or 3′ from the orphan nucleotide comprises a ribose with a 2′-OH group, or a deoxyribose with a 2′-H group, or a mixture of these two. The Central Triplet with the orphan nucleotide in the middle then consists of DNA-DNA-DNA; DNA-DNA-RNA; DNA-RNA-DNA; DNA-RNA-RNA; RNA-DNA-DNA; RNA-DNA-RNA; RNA-RNA-DNA; or RNA-RNA-RNA; wherein the orphan nucleotide does not have a 2′-OMe modification (when RNA) and either or both surrounding nucleotides also do not have a 2′-OMe modification. It is then preferred that all other nucleotides in the AON then do have a 2′-O-alkyl group, preferably a 2′-OMe group, or a 2′-MOE group, or any modification as disclosed herein. Wobbles, mismatches and/or bulges of the AON of the present invention with the target sequence do not prevent hybridization of the oligonucleotide to the target RNA sequence, but add to the RNA editing efficiency by the ADAR present in the cell, at the target adenosine position. The person skilled in the art can determine whether hybridization under physiological conditions still does take place. The AON of the present invention can recruit (engage) a mammalian ADAR enzyme present in the cell, wherein the ADAR enzyme comprises its natural dsRNA binding domain as found in the wild type enzyme. The AONs according to the present invention can utilise endogenous cellular pathways and a naturally available ADAR enzyme, or enzymes with ADAR activity (which may be yet unidentified ADAR-like enzymes) to specifically edit a target adenosine in a target RNA sequence. As disclosed herein, the single-stranded AONs of the invention are capable of deamination of a specific target, such as adenosine, in a target RNA molecule, even though they are significantly shorter than what has been shown thus far in the field of RNA editing. Ideally, only one nucleotide is deaminated. Alternatively, 1, 2, or 3 further nucleotides are deaminated, but preferably only one. The AONs of the invention can be designed for and used with a variety of nucleotide deaminase enzymes, for example ADAR. The ADAR is preferably naturally expressed but may also be produced artificially (e.g. by recombinant expression or protein synthesis). The ADAR can be wild-type or modified. Taking the features of the AONs of the present invention together, there is no need for modified recombinant ADAR expression. The AONs of the invention are not particularly limited regarding conjugated entities attached to the AON. However, there is no need for conjugated entities attached to the AON. As such, AONs lacking conjugated entities attached to the AON form a preferred embodiment. There is no need for the presence of long recruitment portions that are not complementary to the target RNA sequence. Consequently, AONs lacking long recruitment portions that are not complementary to the target RNA sequence form a preferred embodiment. The AONs of the present invention are smaller than those used in the art for RNA editing, but can still recruit endogenous ADAR, and bring about site-specific RNA editing, as shown herein. The Editing AON of the invention (with or without the assistance of the Helper AON) is capable of engaging an entity, preferably an enzyme, with deamination activity that is preferably endogenously present in a cell, preferably a mammalian, more preferably a human cell, to provide deamination of the target nucleotide in the target nucleic acid molecule. A preferred target nucleic acid molecule is an RNA molecule. The double stranded AON/target nucleic acid molecule complex interacts through Watson-Crick base-pairing. In one preferred aspect of the invention, the AON comprises one or more MP linkages at linkage positions 0, −2, −4, and/or −6. In a particularly preferred embodiment, the AON comprises MP linkages at positions 0 and/or −2. In one preferred aspect, the AON comprises at least one PS, at least one phosphonoacetate internucleotide linkage, and/or at least one nucleotide comprising an unlocked nucleic acid (UNA) ribose modification. In a more preferred aspect PS linkages are present at both termini of the AON, connecting the ultimate three, four or five nucleosides on each end. As outlined herein, beforehand AONs for RNA editing were in the range of 35 to 40 nt in length, but the present invention shows that single AONs that are in the range of 19, 20 and 21 nucleotides are also capable of recruiting ADAR and bring about RNA editing.

In another preferred aspect, the AON of the invention further comprises one or more nucleotides comprising a substitution at the 2′ position of the ribose, wherein the substitution is selected from the group consisting of: —OH; —F; substituted or unsubstituted, linear or branched lower (C1-C10) alkyl, alkenyl, alkynyl, alkaryl, allyl, or aralkyl, that may be interrupted by one or more heteroatoms; —O—, S—, or N-alkyl; —O—, S—, or N-alkenyl; —O—, S—, or N-alkynyl; —O—, S—, or N-allyl; —O-alkyl-O-alkyl; -methoxy; -aminopropoxy; -methoxyethoxy (2′-MOE); -dimethylamino oxyethoxy; and -dimethylaminoethoxyethoxy. The Editing AON according to the present invention is preferably 16, 17, 18, 19, 20, 21, or 22 nucleotides in length, and preferably is 21 nucleotides long. The Helper AON according to the present invention is preferably 16, 17, 18, 19, 20, 21, or 22 nucleotides in length, and preferably is 17 nucleotides long.

In another embodiment, the invention relates to a pharmaceutical composition comprising the AON according to the invention (or a set of two AONs according to the invention), and a pharmaceutically acceptable carrier. Pharmaceutically acceptable carriers are known to the person skilled in the art.

In another embodiment, the invention relates to the use of an AON according to the invention, or a set of two AONs (Editing AON +Helper AON) according to the invention, in the manufacture of a medicament for the treatment of a genetic disorder, preferably selected from the group consisting of: Cystic fibrosis, Hurler Syndrome, alpha-1-antitrypsin (A1AT) deficiency, Parkinson's disease, Alzheimer's disease, albinism, Amyotrophic lateral sclerosis, Asthma, β-thalassemia, CADASIL, Charcot-Marie-Tooth disease, Chronic Obstructive Pulmonary Disease (COPD), Distal Spinal Muscular Atrophy (DSMA), Duchenne/Becker muscular dystrophy, (Dystrophic) Epidermolysis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber's Congenital Amaurosis (such as LCA10), Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-esol related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anemia, Spinal Muscular Atrophy, Stargardt disease, Tay-Sachs Disease, Usher syndrome (such as Usher syndrome type I, type II, and type III), X-linked immunodeficiency, Sturge-Weber Syndrome, and cancer.

In yet another embodiment, the invention relates to a method for the deamination of at least one target nucleotide, preferably an adenosine, present in a target nucleic acid molecule, preferably an RNA target molecule, in a cell, the method comprising the steps of: providing the cell with an Editing AON according to the invention, a set of two AONs (Editing AON+Helper AON) according to the invention, or the pharmaceutical composition according to the invention; allowing annealing of the AON(s) to the target nucleic acid molecule; allowing a mammalian enzyme with nucleotide deaminase activity to deaminate the target nucleotide in the target nucleic acid molecule; and optionally identifying the presence of the deaminated nucleotide in the target nucleic acid molecule. The mammalian enzyme with nucleotide deaminase activity that is engaged through the use of the AON according to the invention is preferably an adenosine deaminase enzyme, and is capable of altering the target nucleotide in the target nucleic acid molecule, which target nucleotide is then preferably an adenosine that is deaminated to an inosine. The optional step of identifying the presence of the deaminated nucleotide is preferably performed by: sequencing a region of the target nucleic acid molecule, wherein the region comprises the deaminated target nucleotide; assessing the presence of a functional, elongated, full length and/or wild type protein when the target nucleotide is an adenosine located in a UGA or UAG stop codon, which is edited to a UGG codon through the deamination; assessing the presence of a functional, elongated, full length and/or wild type protein when two target adenosines are located in a UAA stop codon, which is edited to a UGG codon through the deamination of both target adenosines; assessing, when the target RNA molecule is pre-mRNA, whether splicing of the pre-mRNA was altered by the deamination; or using a functional read-out, wherein the target nucleic acid molecule after the deamination encodes a functional, full length, elongated and/or wild type protein.

In another embodiment, the invention relates to a method of treating a subject, preferably a human subject in need thereof, wherein the subject suffers from a genetic disorder caused by a mutation involving the appearance of an adenosine (for instance in a PTC), and in which deamination of that adenosine to an inosine would alleviate, prevent, or ameliorate the disease, comprising the steps of administering to the subject an Editing AON, or a set of two AONs (Editing AON+Helper AON) according to the invention, or a pharmaceutical composition according to the invention, allowing the formation of a double stranded nucleic acid complex of the AON with its specific complementary target nucleic acid in a cell in the subject; allowing the engagement of an endogenous present enzyme with deamination activity, such as hADAR1 or hADAR2; and allowing the enzyme to deaminate the target adenosine in the target nucleic target molecule to an inosine, thereby alleviating, preventing or ameliorating the genetic disease. The genetic diseases that may be treated according to this method are preferably, but not limited to the genetic diseases listed herein (see above).

### Definitions

The terms ‘adenine’, ‘guanine’, ‘cytosine’, ‘thymine’, ‘uracil’ and ‘hypoxanthine’ (the nucleobase in inosine) as used herein refer to the nucleobases as such. The terms ‘adenosine’, ‘guanosine’, ‘cytidine’, ‘thymidine’, ‘uridine’ and ‘inosine’ refer to the nucleobases linked to the (deoxy)ribosyl sugar. The term ‘nucleoside’ refers to the nucleobase linked to the (deoxy)ribosyl sugar, without phosphate groups. A ‘nucleotide’ is composed of a nucleoside and one or more phosphate groups. The term ‘nucleotide’ thus refers to the respective nucleobase-(deoxy)ribosyl-phospholinker, as well as any chemical modifications of the ribose moiety or the phospho group. Thus the term would include a nucleotide including a locked ribosyl moiety (comprising a 2′-4′ bridge, comprising a methylene group or any other group), an unlocked nucleic acid (UNA), a nucleotide including a linker comprising a phosphodiester, phosphonoacetate, phosphotriester, phosphorothioate (PS), phosphoro(di)thioate, methyl(ene)phosphonate (MP), phosphoramidate linkers, and the like. Sometimes the terms adenosine and adenine, guanosine and guanine, cytidine and cytosine, uracil and uridine, thymine and thymidine/uridine, inosine and hypo-xanthine, are used interchangeably to refer to the corresponding nucleobase on the one hand, and the nucleoside or nucleotide on the other. Sometimes the terms nucleobase, nucleoside and nucleotide are used interchangeably, unless the context clearly requires differently, for instance when a nucleoside is linked to a neighbouring nucleoside and the linkage between these nucleosides is modified. In this case, it may be considered that the nucleoside has a modified linker, or that the nucleotide is a modified nucleotide. As stated above, a nucleotide is a nucleoside+one or more phosphate groups. The terms ‘ribonucleoside’ and ‘deoxyribonucleoside’, or ‘ribose’ and ‘deoxyribose’ are as used in the art. Whenever reference is made to an ‘antisense oligonucleotide’, ‘oligonucleotide’, ‘EON’, or ‘AON’ both oligoribonucleotides and deoxyoligoribonucleotides are meant unless the context dictates otherwise. Whenever reference is made to an ‘oligoribonucleotide’ it may comprise the bases A, G, C, U or I. Whenever reference is made to a ‘deoxyoligoribonucleotide’ it may comprise the bases A, G, C, T or I.

In a preferred aspect, the AON of the present invention is an oligoribonucleotide that may comprise chemical modifications and may include deoxynucleotides (DNA) at certain specified positions. Terms such as oligonucleotide, oligo, ON, oligonucleotide composition, antisense oligonucleotide, AON, (RNA) editing oligonucleotide, EON, and RNA (antisense) oligonucleotide may be used herein interchangeably. Whenever reference is made to nucleotides in the oligonucleotide construct, such as cytosine, 5-methylcytosine, 5-hydroxymethylcytosine and β-D-Glucosyl-5-hydroxy-methylcytosine are included; when reference is made to adenine, N6-Methyladenine and 7-methyladenine are included; when reference is made to uracil, dihydrouracil, 4-thiouracil and 5-hydroxymethyluracil are included; when reference is made to guanine, 1-methylguanine is included. Whenever reference is made to nucleosides or nucleotides, ribofuranose derivatives, such as 2′-desoxy, 2′-hydroxy, and 2′-O-substituted variants, such as 2′-O-methyl, are included, as well as other modifications, including 2′-4′ bridged variants. Whenever reference is made to oligonucleotides, linkages between two mononucleotides may be phosphodiester linkages as well as modifications thereof, including, phosphonoacetate, phosphodiester, phosphotriester, PS, phosphoro(di)thioate, MP, phosphor-amidate linkers, and the like.

The term ‘comprising’ encompasses ‘including’ as well as ‘consisting’, e.g. a composition ‘comprising X’ may consist exclusively of X or may include something additional, e.g. X+Y. The term ‘about’ in relation to a numerical value x is optional and means, e.g. x±10%. The word ‘substantially’ does not exclude ‘completely’, e.g. a composition which is ‘substantially free from Y’ may be completely free from Y. Where relevant, the word ‘substantially’ may be omitted from the definition of the invention.

The term “complementary” as used herein refers to the fact that the AON hybridizes under physiological conditions to the target sequence. The term does not mean that each nucleotide in the AON has a perfect pairing with its opposite nucleotide in the target sequence. In other words, while an AON may be complementary to a target sequence, there may be mismatches, wobbles and/or bulges between AON and the target sequence, while under physiological conditions that AON still hybridizes to the target sequence such that the cellular RNA editing enzymes can edit the target adenosine. The term “substantially complementary” therefore also means that in spite of the presence of the mismatches, wobbles, and/or bulges, the AON has enough matching nucleotides between AON and target sequence that under physiological conditions the AON hybridizes to the target RNA. As shown herein, an AON may be complementary, but may also comprise one or more mismatches, wobbles and/or bulges with the target sequence, if under physiological conditions the AON is able to hybridize to its target.

The term ‘downstream’ in relation to a nucleic acid sequence means further along the sequence in the 3′ direction; the term ‘upstream’ means the converse. Thus, in any sequence encoding a polypeptide, the start codon is upstream of the stop codon in the sense strand but is downstream of the stop codon in the antisense strand.

References to ‘hybridisation’ typically refer to specific hybridisation and exclude non-specific hybridisation. Specific hybridisation can occur under experimental conditions chosen, using techniques well known in the art, to ensure that the majority of stable interactions between probe and target are where the probe and target have at least 70%, preferably at least 80%, more preferably at least 90% sequence identity. The term ‘mismatch’ is used herein to refer to opposing nucleotides in a double stranded RNA complex which do not form perfect base pairs according to the Watson-Crick base pairing rules. Mismatched nucleotides are G-A, C-A, U-C, A-A, G-G, C-C, U-U pairs. In some embodiments AONs of the present invention comprise fewer than four mismatches, for example 0, 1 or 2 mismatches. Wobble base pairs are: G-U, I-U, I-A, and I-C base pairs.

An AON, more in particular the Editing AON, of the present invention comprises a nucleotide that is directly opposite the target nucleotide present in the target RNA molecule. The nucleotide in the AON that is directly opposite the target nucleotide is herein defined as the ‘orphan nucleotide’. The ‘Central Triplet’ is defined as the region within the AON consisting of the orphan nucleotide plus its 3′ and 5′ neighbouring nucleotides (hence, the Central Triplet =three nucleotides with the orphan nucleotide in the middle).

The term ‘splice mutation’ relates to a mutation in a gene that encodes for a pre-mRNA, wherein the splicing machinery is dysfunctional in the sense that splicing of introns from exons is disturbed and due to the aberrant splicing the subsequent translation is out of frame resulting in premature termination of the encoded protein. Often such shortened proteins are degraded rapidly and do not have any functional activity, as discussed herein. The exact mutation does not have to be the target for the RNA editing; it may be that a neighbouring or nearby adenosine in the splice mutation is the target nucleotide, which conversion to I fixes the splice mutation back to a normal state. The skilled person is aware of methods to determine whether normal splicing is restored, after RNA editing of the adenosine within the splice mutation site or area.

An AON according to the present invention may be chemically modified at almost its entirety of nucleosides, for example by providing nucleosides with a 2′-O-methylated sugar moiety (2′-OMe) and/or with a 2′-O-methoxyethyl sugar moiety (2′-MOE). However, the orphan nucleotide preferably does not comprise the 2′-OMe modification, and in yet a further preferred aspect, at least one and in a preferred aspect both the two neighbouring nucleotides flanking each nucleotide opposing the target adenosine further do not comprise a 2′-OMe modification. Complete modification wherein all nucleotides of the AON hold a 2′-OMe modification results in a non-functional oligonucleotide as far as RNA editing goes (known in the art), presumably because it hinders the ADAR activity at the targeted position. In general, an adenosine in a target RNA can be protected from editing by providing an opposing nucleotide with a 2′-OMe group, or by providing a guanine or adenine as opposing base, as these two nucleobases are also able to reduce editing of the opposing adenosine. Various chemistries and modification are known in the field of oligonucleotides that can be readily used in accordance with the invention. The regular internucleosidic linkages between the nucleotides may be altered by mono- or di-thioation of the phosphodiester bonds to yield phosphorothioate esters or phosphorodithioate esters, respectively. Other modifications of the internucleosidic linkages are possible, including amidation and peptide linkers. MP linkages can be formed using known chemistries.

It is known in the art, that RNA editing entities (such as human ADAR enzymes) edit dsRNA structures with varying specificity, depending on several factors. One important factor is the degree of complementarity of the two strands making up the dsRNA sequence. Perfect complementarity of the two strands usually causes the catalytic domain of hADAR to deaminate adenosines in a non-discriminative manner, reacting with any adenosine it encounters. The specificity of hADAR1 and 2 can be increased by introducing chemical modifications and/or ensuring a number of mismatches in the dsRNA, which presumably help to position the dsRNA binding domains in a way that has not been clearly defined yet. Additionally, the deamination reaction itself can be enhanced by providing an AON that comprises a mismatch opposite the adenosine to be edited. The mismatch is preferably created by providing a targeting portion having a cytidine opposite the adenosine to be edited. As an alternative, also uridines may be used opposite the adenosine, which, understandably, will not result in a ‘mismatch’ because U and A pair. Upon deamination of the adenosine in the target strand, the target strand will obtain an inosine which, for most biochemical processes, is “read” by the cell's biochemical machinery as a G. Hence, after A to I conversion, the mismatch has been resolved, because I is perfectly capable of base pairing with the opposite C in the targeting portion of the oligonucleotide construct according to the invention. After the mismatch has been resolved due to editing, the substrate is released and the oligonucleotide construct-editing entity complex is released from the target RNA sequence, which then becomes available for downstream biochemical processes, such as splicing and translation. Also, this on/off rate is important because the targeting oligonucleotide should not be too tightly bound to the target RNA. The desired level of specificity of editing the target RNA sequence may depend from target to target. Following the instructions in the present patent application, those of skill in the art will be capable of designing the complementary portion of the oligonucleotide according to their needs, and, with some trial and error, obtain the desired result.

RNA editing molecules present in the cell will usually be proteinaceous in nature, such as the ADAR enzymes found in metazoans, including mammals. Preferably, the cellular editing entity is an enzyme, more preferably an adenosine deaminase or a cytidine deaminase, still more preferably an adenosine deaminase. These are enzymes with ADAR activity. The ones of most interest are the human ADARs, hADAR1 and hADAR2, including any isoforms thereof. RNA editing enzymes known in the art, for which oligonucleotide constructs according to the invention may conveniently be designed, include the adenosine deaminases acting on RNA (ADARs), such as hADAR1 and hADAR2 in humans or human cells and cytidine deaminases. It is known that hADAR1 exists in two isoforms; a long 150 kDa interferon inducible version and a shorter, 100 kDa version, that is produced through alternative splicing from a common pre-mRNA. Consequently, the level of the 150 kDa isoform available in the cell may be influenced by interferon, particularly interferon-gamma (IFN-γ). hADAR1 is also inducible by TNF-α. This provides an opportunity to develop combination therapy, whereby IFN-γ or TNF-α and AONs according to the invention are administered to a patient either as a combination product, or as separate products, either simultaneously or subsequently, in any order. Certain disease conditions may already coincide with increased IFN-γ or TNF-α levels in certain tissues of a patient, creating further opportunities to make editing more specific for diseased tissues. It will be understood by a person having ordinary skill in the art that the extent to which the editing entities inside the cell are redirected to other target sites may be regulated by varying the affinity of the AONs according to the invention for the recognition domain of the editing molecule. The exact modification may be determined through some trial and error and/or through computational methods based on structural interactions between the AON and the recognition domain of the editing molecule. In addition, or alternatively, the degree of recruiting and redirecting the editing entity resident in the cell may be regulated by the dosing and the dosing regimen of the AON. This is something to be determined by the experimenter (in vitro) or the clinician, usually in clinical trials.

The invention concerns the modification of target RNA sequences in eukaryotic, preferably metazoan, more preferably mammalian, most preferably human cells. The invention can be used with cells from any organ e.g. skin, lung, heart, kidney, liver, pancreas, gut, muscle, gland, eye, brain, blood, and the like. The invention is particularly suitable for modifying sequences in cells, tissues or organs implicated in a diseased state of a (human) subject. The cell can be located in vitro, ex vivo or in vivo. One advantage of the invention is that it can be used with cells in situ in a living organism, but it can also be used with cells in culture. In some embodiments, cells are treated ex vivo and are then introduced into a living organism (e.g. re-introduced into an organism from whom they were originally derived). The invention can also be used to edit target RNA sequences in cells within a so-called organoid. Organoids can be thought of as three-dimensional in vitro-derived tissues but are driven using specific conditions to generate individual, isolated tissues. In a therapeutic setting they are useful because they can be derived in vitro from a patient's cells, and the organoids can then be re-introduced to the patient as autologous material which is less likely to be rejected than a normal transplant. The cell to be treated will generally have a genetic mutation. The mutation may be heterozygous or homozygous. The invention will typically be used to modify point mutations, such as N to A mutations, wherein N may be G, C, U (on the DNA level T), preferably G to A mutations, or N to C mutations, wherein N may be A, G, U (on the DNA level T), preferably U to C mutations.

Without wishing to be bound be theory, the RNA editing through hADAR1 and hADAR2 is thought to take place on primary transcripts in the nucleus, during transcription or splicing, or in the cytoplasm, where e.g. mature mRNA, miRNA or ncRNA can be edited. Different isoforms of the editing enzymes are known to localize differentially, e.g. with the 100kDa isoform of hADAR1 found mostly in the nucleus, and the 150 kDa isoform of hADAR1 in the cytoplasm. The RNA editing by cytidine deaminases is thought to take place on the mRNA level.

Many genetic diseases are caused by G to A mutations, and these are preferred target diseases because adenosine deamination at the mutated target adenosine will reverse the mutation to a codon giving rise to a functional, full length and/or wild type protein, especially when it concerns PTCs. Preferred examples of genetic diseases that can be prevented and/or treated with oligonucleotides according to the invention are any disease where the modification of one or more adenosines in a target RNA will bring about a (potentially) beneficial change. Especially preferred are Usher syndrome, Stargardt disease and Cystic Fibrosis.

It should be clear, that targeted editing according to the invention can be applied to any adenosine (or cytosine), whether it is a mutated or a wild-type nucleotide. For example, editing may be used to create RNA sequences with different properties. Such properties may be coding properties (creating proteins with different sequences or length, leading to altered protein properties or functions), or binding properties (causing inhibition or over-expression of the RNA itself or a target or binding partner; entire expression pathways may be altered by recoding miRNAs or their cognate sequences on target RNAs). Protein function or localization may be changed at will, by functional domains or recognition motifs, including but not limited to signal sequences, targeting or localization signals, recognition sites for proteolytic cleavage or co- or post-translational modification, catalytic sites of enzymes, binding sites for binding partners, signals for degradation or activation and so on. These and other forms of RNA and protein “engineering”, whether or not to prevent, delay or treat disease or for any other purpose, in medicine or biotechnology, as diagnostic, prophylactic, therapeutic, research tool or otherwise, are encompassed by the present invention.

The amount of AON to be administered, the dosage and the dosing regimen can vary from cell type to cell type, the disease to be treated, the target population, the mode of administration (e.g. systemic versus local), the severity of disease and the acceptable level of side activity, but these can and should be assessed by trial and error during in vitro research, in pre-clinical and clinical trials. The trials are particularly straightforward when the modified sequence leads to an easily detected phenotypic change. It is possible that higher doses of AON could compete for binding to a nucleic acid editing entity (e.g. ADAR) within a cell, thereby depleting the amount of the entity which is free to take part in RNA editing, but routine dosing trials will reveal any such effects for a given AON and a given target.

One suitable trial technique involves delivering the AON(s) to cell lines, or a test organism and then taking biopsy samples at various time points thereafter. The sequence of the target RNA can be assessed in the biopsy sample and the proportion of cells having the modification can easily be followed. After this trial has been performed once then the knowledge can be retained, and future delivery can be performed without needing to take biopsy samples. A method of the invention can thus include a step of identifying the presence of the desired change in the cell's target RNA sequence, thereby verifying that the target RNA sequence has been modified. This step will typically involve sequencing of the relevant part of the target RNA, or a cDNA copy thereof (or a cDNA copy of a splicing product thereof, in case the target RNA is a pre-mRNA), as discussed above, and the sequence change can thus be easily verified. Alternatively the change may be assessed on the level of the protein (length, glycosylation, function or the like), or by some functional read-out, such as a(n) (inducible) current, when the protein encoded by the target RNA sequence is an ion channel, for example.

After RNA editing has occurred in a cell, the modified RNA can become diluted over time, for example due to cell division, limited half-life of the edited RNAs, etc. Thus, in practical therapeutic terms a method of the invention may involve repeated delivery of an AON until enough target RNAs have been modified to provide a tangible benefit to the patient and/or to maintain the benefits over time.

AONs of the invention are particularly suitable for therapeutic use, and so the invention provides a pharmaceutical composition comprising an AON of the invention and a pharmaceutically acceptable carrier. In some embodiments of the invention the pharmaceutically acceptable carrier can simply be a saline solution. This can usefully be isotonic or hypotonic, particularly for pulmonary delivery. The invention also provides a delivery device (e.g. syringe, inhaler, nebuliser) which includes a pharmaceutical composition of the invention.

The invention also provides an AON of the invention for use in a method for making a change in a target RNA sequence in a mammalian, preferably a human cell, as described herein. Similarly, the invention provides the use of an AON of the invention in the manufacture of a medicament for making a change in a target RNA sequence in a mammalian, preferably a human cell, as described herein.

The invention also relates to a method for the deamination of at least one specific target adenosine present in a target RNA sequence in a cell, the method comprising the steps of: providing the cell with an AON according to the invention; allowing uptake by the cell of the AON; allowing annealing of the AON to the target RNA molecule; allowing a mammalian ADAR enzyme comprising a natural dsRNA binding domain as found in the wild type enzyme to deaminate the target adenosine in the target RNA molecule to an inosine; and optionally identifying the presence of the inosine in the RNA sequence.

In a preferred aspect, depending on the ultimate deamination effect of A to I conversion, the identification step comprises: sequencing the target RNA; assessing the presence of a functional, elongated, full length and/or wild type protein; assessing whether splicing of the pre-mRNA was altered by the deamination; or using a functional read-out, wherein the target RNA after the deamination encodes a functional, full length, elongated and/or wild type protein. Because the deamination of the adenosine to an inosine may result in a protein that is no longer suffering from the mutated A at the target position, the identification of the deamination into inosine may also be a functional read-out, for instance an assessment on whether a functional protein is present, or even the assessment that a disease that is caused by the presence of the adenosine is (partly) reversed. The functional assessment for each of the diseases mentioned herein will generally be according to methods known to the skilled person. A very suitable manner to identify the presence of an inosine after deamination of the target adenosine is of course RT-PCR and sequencing, using methods that are well-known to the person skilled in the art.

The AON(s) according to the invention is (are) suitably administrated in aqueous solution, e.g. saline, or in suspension, optionally comprising additives, excipients and other ingredients, compatible with pharmaceutical use, at concentrations ranging from 1 ng/ml to 1 g/ml, preferably from 10 ng/ml to 500 mg/ml, more preferably from 100 ng/ml to 100 mg/ml. Dosage may suitably range from between about 1 μg/kg to about 100 mg/kg, preferably from about 10 μg/kg to about 10 mg/kg, more preferably from about 100 μg/kg to about 1 mg/kg. Administration may be by inhalation (e.g. through nebulization), intranasally, orally, by injection or infusion, intravenously, subcutaneously, intra-dermally, intra-cranially, intravitreally, intramuscularly, intra-tracheally, intra-peritoneally, intra-rectally, and the like. Administration may be in solid form, in the form of a powder, a pill, a gel, an eye-drop, or in any other form compatible with pharmaceutical use in humans.

### EXAMPLES

### Example 1. Design of Split Antisense Oligonucleotides for A to I Editing

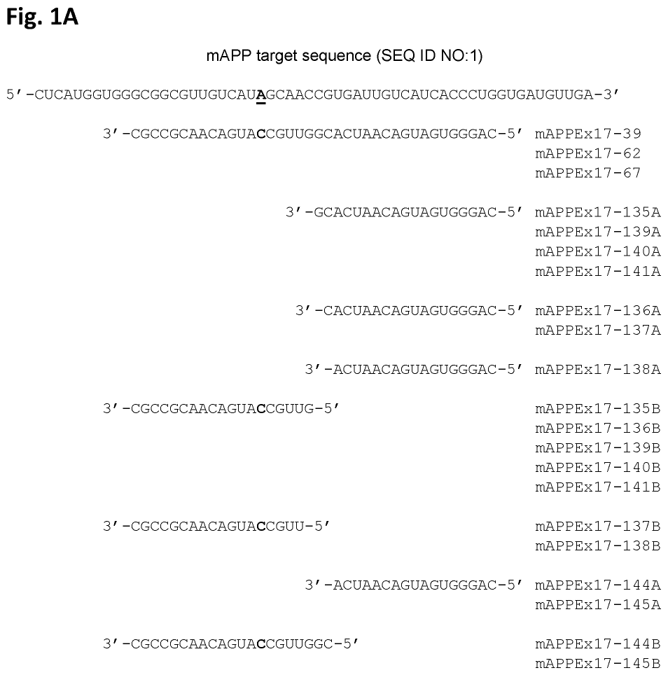

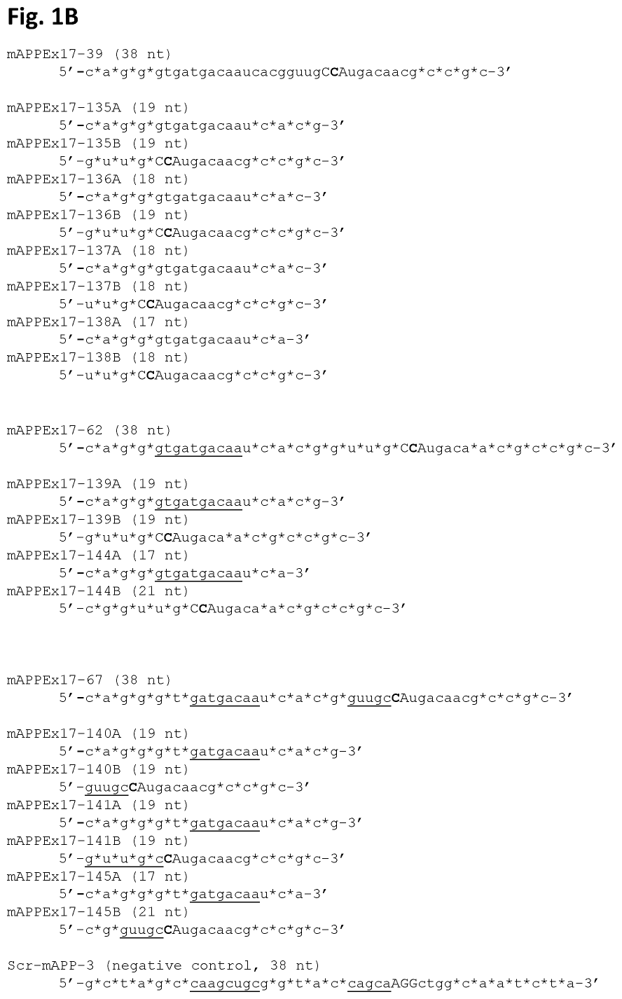

The inventors of the present invention realized that a single 38 nt long RNA editing antisense oligonucleotide (AON) could give problems in respect of manufacturing as well as in efficiency of RNA editing in vivo. It was conceived as uncertain how efficient such relatively ‘large’ AONs could enter cells in vivo, without the use of transfection agents. Notably, the art does not mention a solution for this and the inventors then investigated the use of two short AONs in which one would represent the 5′ part of the longer AON and the other would represent the 3′ part of the longer version. The AON representing the 5′ part would be the ‘Helper AON’, whereas the AON representing the 3′ part would be considered the ‘Editing AON’ because it would comprise the ‘orphan nucleotide’ opposite the adenosine that would be the target for editing in the target RNA molecule. As a model, the (pre-) mRNA that codes for the mouse Amyloid Precursor Protein (mAPP) was selected as the target. FIG. 1A shows the sequence of the target mAPP mRNA as well as three 38-nt long AONs referred to as mAPPEx17-39, -62, and -67, that all served as positive controls, albeit with different chemical modifications (FIG. 1B). It was initially found that mAPPex17-62 was able to induce editing of the adenosine in the target sequence (bold, underlined in FIG. 1A) in exon 17 of mAPP wild-type (pre-) mRNA (data not shown). As mentioned, a length of 38 nucleotides is very common for most RNA editing AONs used in the field of RNA editing. The inventors decided to split the sequence of this AON in two separate parts, first by splitting the 38 nt AON sequence symmetrically by generating four different sets of AONs with each AON being 19 nt long, then also by splitting the 38 nt AON sequence asymmetrically, and by splitting the 38 nt AON sequence in two parts leaving a gap of 1, 2 or 3 nucleotides between the shorter AONs, as follows:

### Symmetrical Split

mAPPEx17-135A (19 nt) and mAPPEx17-135B (19 nt)
mAPPEx17-139A (19 nt) and mAPPEx17-139B (19 nt)
mAPPEx17-140A (19 nt) and mAPPEx17-140B (19 nt)
mAPPEx17-141A (19 nt) and mAPPEx17-141B (19 nt)

### Asymmetrical Split

mAPPEx17-144A (17 nt) and mAPPEx17-144B (21 nt)
mAPPEx17-145A (17 nt) and mAPPEx17-145B (21 nt)

### Gapped Split

mAPPEx17-136A (18 nt) and mAPPEx17-136B (19 nt) — 1 nt gap
mAPPEx17-137A (18 nt) and mAPPEx17-139B (18 nt) — 2 nt gap
mAPPEx17-138A (17 nt) and mAPPEx17-138B (18 nt) — 3 nt gap

The AONs denoted with an ‘A’ are also referred to as “Helper AONs” and the AONs denoted with a ‘B’ are also referred to as “Editing AONs”. The AONs carried a variety of chemical modifications in line with the single 38 nt AONs that were used as positive controls in each experiment. The AONs carried a variety of RNA/DNA, PS linkages, 2′-OMe and/or 2′-MOE modifications (see FIG. 1B for details). All AONs were fully modified at the 2′ position of the sugar moiety, either with 2′-O-methyl or with 2′-O-methoxyethyl (see FIG. 1B), except for the nucleotide (the orphan nucleotide) opposite the to-be-edited A, and its two surrounding nucleotides (one on the 5′ and one on the 3′ side), which three nucleotides were DNA. The number of PS linkages varied as can be seen in FIG. 1B. It should be realized that other asymmetric splits than 17/21 are also feasible (A/B: 18/20, 20/18, 21/17).

### Example 2. Using Sets of Split AONs for A to I Editing in an In Vitro Biochemical Assay

The editing efficacy of all the sets referred to above was measured and compared to a positive control 38 nt AON carrying similar chemical modifications in an in vitro biochemical editing assay.

To obtain the mAPP target RNA a PCR was performed using a mAPP G-block (IDT) which contained the sequence for the T7 promotor and (a part of) the sequence of mAPP as template using forward primer 5′-CTCGACGCAAGCCATAACAC-3′ (SEQ ID NO:10) and reverse primer 5′-TGGACCGACTGGAAACGTAG-3′ (SEQ ID NO:11). The PCR product was then used as template for the in vitro transcription. The MEGAscript T7 transcription kit was used for this reaction. The RNA was purified on a urea gel then extracted in 50 mM Tris-Cl pH 7.4, 10 mM EDTA, 0.1% SDS, 0.3 M NaCl buffer and phenol-chloroform purified. The purified RNA was used as target in the biochemical editing assay.

All single AONs and sets of symmetric/asymmetric/gapped AONs were annealed to the mAPP target RNA in a buffer (5 mM Tris-Cl pH 7.4, 0.5 mM EDTA and 10 mM NaCl) at the ratio 1:3 of target RNA to AON (200 nM target RNA and 600 nM AON). The samples were heated at 95° C. for 3 min and then slowly cooled down to RT. Next, the editing reaction was carried out. The double stranded nucleic acid complex was mixed with protease inhibitor (cOmplete™, Mini, EDTA-free Protease I, Sigma-Aldrich), RNase inhibitor (RNasin, Promega), poly A (Qiagen), tRNA (Invitrogen) and editing reaction buffer (15 mM Tris-Cl pH 7.4, 1.5 mM EDTA, 3% glycerol, 60 mM KCl, 0.003% NP-40, 3 mM MgCl₂ and 0.5 mM DTT) such that their final concentration was 6 nM AON and 2 nM target RNA. The reaction was started by adding purified ADAR2 (GenScript) to a final concentration of 6 nM into the mix. Incubation lasted for predetermined times (0 s, 30 s, 1 min, 2 min, 5 min, 10 min, 25 min and 50 min) at 37° C. Each reaction was stopped by adding 95 μl of boiling 3 mM EDTA solution.

A 6 μl aliquot of the stopped reaction mixture was then used as template for cDNA synthesis using Maxima reverse transcriptase kit (Thermo Fisher) with a random hexamer primer (ThermoFisher Scientific). Initial denaturation of RNA was performed in the presence of the primer and dNTPs at 95° C. for 5 min, followed by slow cooling to 10° C., after which first strand synthesis was carried out according to the manufacturer's instructions in a total volume of 20 μl, using an extension temperature of 62° C.

Products were amplified for pyrosequencing analysis by PCR, using the Amplitaq gold 360 DNA Polymerase kit (Applied Biosystems) according to the manufacturer's instructions, with 1 μl of the cDNA as template. The following primers were used at a concentration of 10 μM: Pyroseq Fwd mAPP, 5′-AACTGGTGTTCTTTGCTGAAGAT-3′ (SEQ ID NO:12), and Pyroseq Rev mAPP Biotin, 5′-/5BiosG/CATGATGGATGGATGTGTACTGT-3′ (SEQ ID NO:13). The latter primer also contains a biotin conjugated to its 5′ end, as required for the automatic processing during the pyrosequencing reactions. The PCR was performed using the following thermal cycling protocol: Initial denaturation at 95° C. for 5 min, followed by 40 cycles of 95° C. for 30 s, 58° C. for 30 s and 72° C. for 30 s, and a final extension of 72° C. for 7 min.

As inosines base-pair with cytidines during the cDNA synthesis in the reverse transcription reaction, the nucleotides incorporated in the edited positions during PCR will be guanosines. The percentage of guanosine (edited) versus adenosine (unedited) was defined by pyrosequencing. Pyrosequencing of the PCR products and the following data analysis were performed by the PyroMark Q48 Autoprep instrument (QIAGEN) following the manufacturer's instructions, with 10 μl input of the PCR product and 4 μM of the following sequencing primer: mAPP-Seq, 5′-TCGGACTCATGGTGG-3′ (SEQ ID NO:14). The settings specifically defined for this target RNA strand included two sets of sequence information. The first of these defines the sequence for the instrument to analyse, in which the potential for a position to contain either an adenosine or a guanosine is indicated by an “R” for purine: GCGGCGTTGTCATRGCAACCGTGAT TGTCATCACCCTGGTGATGTTGAAGAAGAA (SEQ ID NO:15). The dispensation order was defined for this analysis as follows: TGCGCGTGTCACTAGCACGTGATGTCATCAC (SEQ ID NO:16). The analysis performed by the instrument provides the results for the selected nucleotide as a percentage of adenosine and guanosine detected in that position, and the extent of A-to-I editing at a chosen position will therefore be measured by the percentage of guanosine in that position.

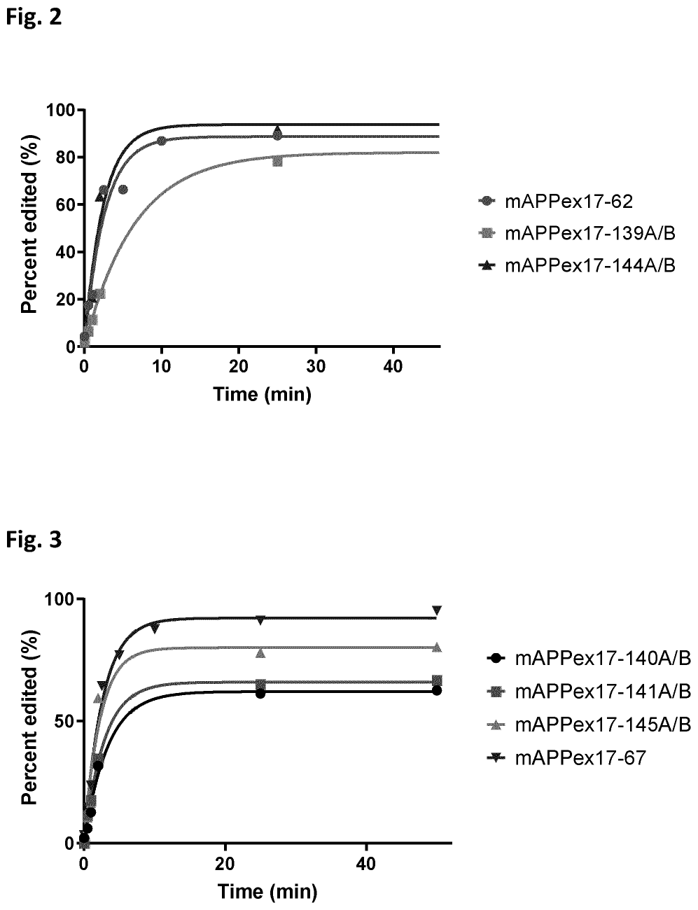

In a first experiment a single 38 nt AON mAPPEx17_62 was compared to two sets: i) a symmetrical set comprising mAPPEx17_139A and -B, and ii) an asymmetrical set comprising mAPPEx17_144A and -B. FIG. 2 shows the result of this experiment. It was surprisingly found that a combination of two short AONs can be applied for proper RNA editing, with the asymmetrical set outperforming the positive control AON. The symmetrical set of AONs also provided significant RNA editing, albeit a somewhat slower rate than the long AON and the asymmetrical set.

In a second experiment a single 38 nt AON mAPPEx17_67 was compared to three sets: i) a symmetrical set comprising mAPPEx17_140A and -B, ii) a symmetrical set comprising mAPPEx17_141A and -B, and iii) an asymmetrical set comprising mAPPEx17_145A and -B. FIG. 3 shows the result of this experiment. It was found that all three sets could be applied for proper RNA editing, and again, that the asymmetrical set performed better than the two symmetrical sets, although all sets gave good results. The higher editing efficacy of asymmetrical split AONs may be due to (a preferred) positioning of the split based on structural data from ADAR2/RNA complexes (not shown) and the RBD2 region of ADAR2. To the best of their knowledge, the inventors of the present invention were the first to show that RNA editing could be achieved with two oligonucleotides with a relatively short length of 16 to 22 nt. It is of course clear to the skilled person that ‘asymmetrical’ in this case means that the length of the split AONs is based on an original length of 38 nucleotides of the control AON, and that when that basic sequence is for instance 34 nucleotides (with 4 nucleotides cut from the 3′ end of the long AON), two split AONs will be equal in length (each being 17 nt long) and then be regarded as ‘symmetrical’. The same holds true for a positive control AON that would be for instance 36 nucleotides in length. Clearly, when the positive control would for instance be 37 nt or 35 nt in length, the split will always be ‘asymmetrical’. Hence, the terms ‘symmetrical’ and ‘asymmetrical’ as used herein is solely based on splitting the original positive control AON in a variety of ways and that the terms are therefore somewhat arbitrarily applied.

### Example 3. Using Sets of Split AONs and Single Short Editing AONs for A to I Editing in an In Vitro Biochemical Assay

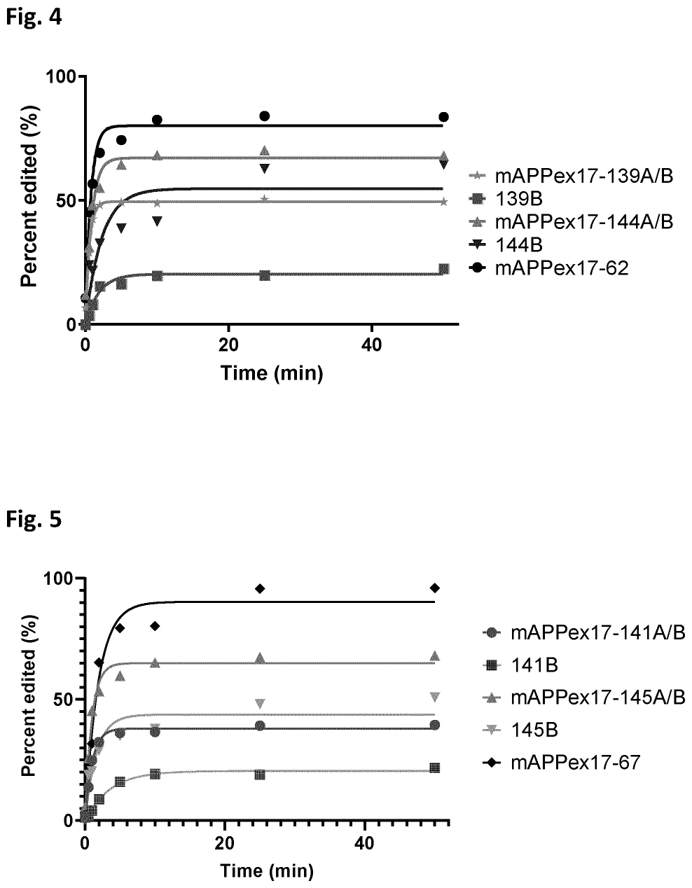

In a similar experimental setup as described above, it was then investigated whether it would also be feasible to use only a single short Editing AON (the ‘B’ versions of the split AONs) for RNA editing without applying the Helper AON (the ‘A’ versions of the split AONs). For this, a single 38 nt AON mAPPEx17_67 was compared to two sets of AONs and two single short Editing AONs, as follows: i) a symmetrical set comprising mAPPEx17_139A and -B, ii) a single Editing AON mAPPEx17_139B, iii) an asymmetrical set comprising mAPPEx17_144A and -B, and iv) a single Editing AON mAPPEx17_144B. FIG. 4 shows the results of this experiment. It was surprisingly found that the use of the single mAPPEx17_144B AON (21 nt) resulted in significant RNA editing that was almost reached levels that were obtained with the same AON together with its Helper AON and with the positive control. This shows that the inventors were able to obtain RNA editing with an AON that is as short as 21 nucleotides, which in view of what has thus far been seen in the field is very remarkable. In this experiment, this single short AON reached even higher levels of RNA editing than the set of mAPP139A and -B used together. The efficacy was further boosted when Editing AON mAPPEx17_144B was used in a set together with its Helper AON mAPPEx17_144A.

In yet a further experiment, a single 38 nt mAPPEx17_67 was compared to two sets of AONs and two other single short Editing AONs, as follows: i) a symmetrical set comprising mAPPEx17_141A and -B, ii) a single Editing AON mAPPEx17_141B, iii) an asymmetrical set comprising mAPPEx17_145A and -B, and iv) a single Editing AON mAPPEx17_145B. FIG. 5 shows the results of this experiment. While the symmetrical set of mAPPEx17_141A and -B did not perform well, the single short mAPPEx17_145B performed better (although not extremely efficacious), which was further boosted by the addition of the Helper AON mAPPEx17_145A, once again showing that the asymmetrical set (as generated in view of the positive control) works better than the symmetrical set of AONs.

Although these experiments reveal that the asymmetrical split AONs and the 21 nt Editing AONs, when used alone, give proper RNA editing results, the variation that is observed between the different single AONs in comparison to the long positive control and the presence of a Helper AON is likely due to exact length, the start of the AON at the 5′ and/or 3′ location and the chemical modifications present in each of these single AONs. Further optimization is needed to provide the best combination of chemical modifications (positions of PS linkages, positions of 2′-OMe and 2′-MOE modifications, the possible introduction of other or additional (2′) modifications at certain positions, the number and/or positions of DNA nucleotides, etc.). It cannot be excluded that length and chemical modifications should be adjusted depending on the specific (pre-) mRNA that is targeted, and therefore also may depend on the specific sequence of the target RNA. In any case, the inventors of the present invention, to the best of their knowledge, were the first to show that RNA editing could be achieved with oligonucleotides that were as short as 19-21 nucleotides in length.

### Example 4. Using Sets of Split AONs and Single Short Editing AONs for A to I Editing in Cells by Recruitment of Endogenous ADAR

Next, it was investigated whether the split AONs (in combination and alone as Editing AONs) could edit wild type endogenous mAPP RNA in cells. For this, mouse retinal pigment epithelium (RPE) cells carrying the wild type APP gene were used. Briefly, 2.5×10⁵ cells per 6 well plate were seeded 24 h before transfection. Transfection was performed with 100 nM AON and Lipofectamine 2000 (Invitrogen) according to the manufacturer's instructions (at a ratio of 1:2, 1 μg AON to 2 μl Lipofectamine 2000). A non-transfection (NT), a mock transfection and a scrambled oligonucleotide (scr-mAPP-3, see FIG. 1B) were taken along as negative controls. RNA was extracted from cells 48 h after transfection using the Direct-zol RNA MiniPrep (Zymo Research) kit according to the manufacturer's instructions, and cDNA prepared using the Maxima reverse transcriptase kit (Thermo Fisher) according to the manufacturer's instructions, with a combination of random hexamer and oligo-dT primers. The cDNA was diluted 5× and 1 μL of this dilution was used as template for digital droplet PCR (ddPCR). The ddPCR assay for absolute quantification of nucleic acid target sequences was performed using BioRad's QX-200 Droplet Digital PCR system. 1 μl of diluted cDNA obtained from the RT cDNA synthesis reaction was used in a total mixture of 21 μl of reaction mix, including the ddPCR Supermix for Probes no dUTP (Bio Rad), a Taqman SNP genotype assay with the following forward and reverse primers combined with the following gene-specific probes:
Forward primer: 5′-CAACATCACCAGGGTGATGAC-3′ (SEQ ID NO:17)
Reverse primer: 5′-CATCATCGGACTCATGGTGG-3′ (SEQ ID NO:18)
Wild type probe (HEX NFQ labeled): 5′-/5HEX/CGT T+GT CAT+A+G+C AAC CGT/3IABkFQ/-3′ (SEQ ID NO:19)
Mutant probe (FAM NFQ labeled): 5′-/56-FAM/CGT TGT CAT+G+G+C AAC CG/3IABkFQ/-3′ (SEQ ID NO:20)

A total volume of 21 μl PCR mix including cDNA was filled in the middle row of a ddPCR cartridge (BioRad) using a multichannel pipette. The replicates were divided by two cartridges. The bottom rows were filled with 70 μl of droplet generation oil for probes (BioRad). After the rubber gasket replacement, droplets were generated in the QX200 droplet generator. 42 μl of oil emulsion from the top row of the cartridge was transferred to a 96-wells PCR plate. The PCR plate was sealed with a tin foil for 4 sec at 170° C. using the PX1 plate sealer, followed by the following PCR program: 1 cycle of enzyme activation for 10 min at 95° C., 40 cycles denaturation for 30 sec at 95° C. and annealing/extension for 1 min at 55.8 ° C., 1 cycle of enzyme deactivation for 10 min at 98° C., followed by a storage at 8° C. After PCR, the plate was read and analysed with the QX200 droplet reader.

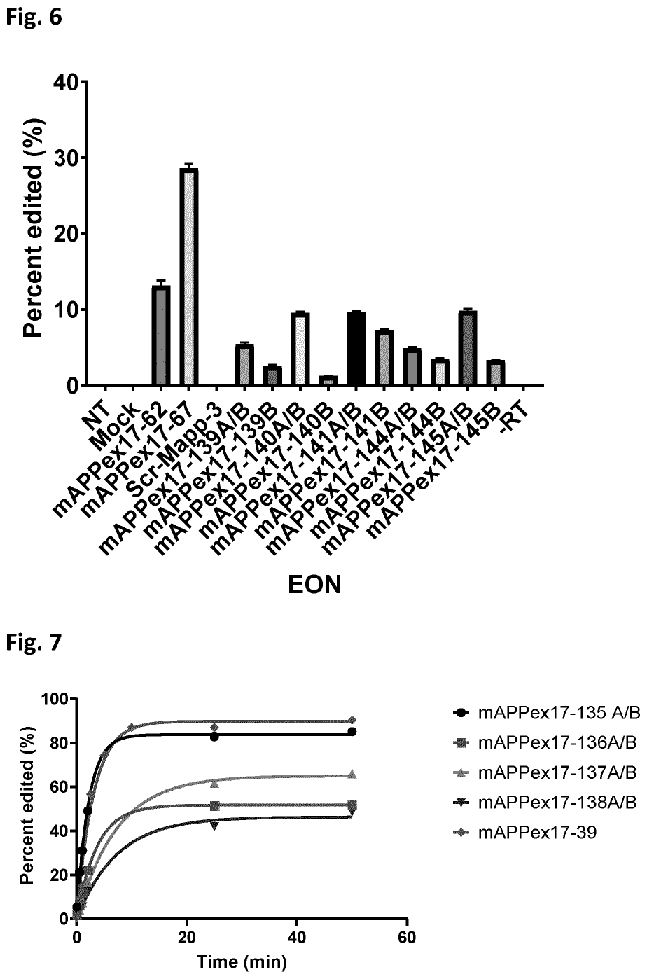

The results shown in FIG. 6 reveal that all Editing AONs used alone or in combination with their respective Helper AONs were capable of editing mAPP target RNA in mouse RPE cells, and able to recruit endogenous ADAR in these cells. mAPPex17-140A+B, mAPPex17-141A+B and mAPPex17-145A+B showed very similar and higher RNA editing levels than the mAPPex17-139A+B and mAPPex17-144A+B sets, albeit with lower efficacy when compared to editing of target RNA by 38 nt long AONs mAPPex17-62 and mAPPex17-67. It should be noted that this experiment made use of transfection reagents, which is different from what happens in vivo, or in vitro when no transfection reagents are used (gymnotic uptake). In transfection the length of the oligonucleotide does not matter much, whereas in the absence of transfection reagents it is known that the longer the oligonucleotide, the harder it is to enter the cell. FIG. 6 also reveals that the single Editing AONs mAPPEx17_139B, -140B, -141B, -144B and -145B all provided RNA editing, which shows that such short versions of RNA editing oligonucleotides are also capable of recruiting endogenous ADAR and are capable of bringing about RNA editing on an endogenous target, which was a striking observation.

### Example 5. Using Sets of Split and Gapped AONs for A to I Editing in an In Vitro Biochemical Assay

The inventors then questioned whether it was necessary that the split AONs should be complementary to a consecutive stretch in the target RNA to give RNA editing, or whether a small gap of 1, 2, or 3 nucleotides between the split AONs would also give proper editing. For this, the same biochemical assays as discussed in Example 2 and 3 was applied. A single 38 nt mAPPEx17_39 was compared to three sets of split (gapped) AONs and one symmetrical set of two consecutive AONs of each 19 nt in length, as follows: i) a symmetrical set comprising mAPPEx17_135A and -B, ii) a 1-nt gap set comprising mAPPEx17_136A and -B, iii) a 2-nt gap set comprising mAPPEx17_137A and -B, and iv) a 3-nt gap set comprising mAPPEx17_138A and -B. For the specific chemical modifications of these AONs, see FIG. 1B. FIG. 7 shows the results of this experiment. All sets showed the ability to bring about RNA editing, indicating that a gap between the two AONs in the sets of oligonucleotides is allowed, but it was also observed that as the gap was present, the efficacy of RNA editing was lowered, indicating that it is preferred that the two split AONs have sequences that provide a full 100% complementarity to a consecutive stretch of nucleotides in the target sequence. Interestingly, the combination of mAPPEx17_135A +B gave a level and speed of RNA editing that was comparable to the level and speed of RNA editing observed with the positive control mAPPEx17_39, once again indicating that the use of two separate oligonucleotides that are in the range of 17-21 nucleotides can be used to obtain deamination of a target adenosine to an inosine in a target (pre-) mRNA molecule.

1. A composition comprising a set of two single stranded antisense oligonucleotides (AONs), wherein one AON is the ‘Editing AON’ and the other AON is the ‘Helper AON’, for use in the deamination of a target adenosine in a target RNA, wherein the Editing AON is complementary to a stretch of nucleotides in the target RNA that includes the target adenosine, wherein the nucleotide in the Editing AON that is directly opposite the target adenosine is the ‘orphan nucleotide’, which is a cytidine that is not modified with 2′-OMe or 2′-MOE, wherein the Helper AON is complementary to a stretch of nucleotides in the target RNA that is separate from the stretch of nucleotides that is complementary to the Editing AON, wherein the Helper AON has a length of 16 to 22 nucleotides and the Editing AON has a length of 16 to 22 nucleotides.

2. The composition according to claim 1, wherein the Helper AON and Editing AON form the double stranded complex with the target RNA in a consecutive manner, wherein the Helper AON is complementary to a stretch of nucleotides in the target RNA that is located at the 3′ side of the stretch of nucleotides in the target RNA that is complementary to the Editing AON, and wherein there is no nucleotide gap between sequences complementary to the Helper AON and the Editing AON.

3. The composition according to claim 1 or 2, wherein the Helper AON is 100% complementary to the target RNA.

4. The composition according to any one of claims 1 to 3, wherein the Editing AON, besides the mismatch between the cytidine opposite the target adenosine, is fully complementary to the target RNA.

5. The composition according to any one of claims 1 to 4, wherein the set, after forming the double stranded complex with the target RNA, is configured to recruit an endogenous ADAR enzyme to bring about the deamination of the target adenosine into an inosine.

6. The composition according to any one of claims 1 to 5, wherein the Editing AON comprises one or more phosphorothioate (PS) linkages.

7. The composition according to any one of claims 1 to 6, wherein the Editing AON comprises at least one nucleotide with a sugar moiety that comprises a 2′-OMe modification, and/or at least one nucleotide with a sugar moiety that comprises a 2′-MOE modification.

8. The composition according to any one of claims 1 to 7, wherein the orphan nucleotide carries a 2′-H in the sugar moiety (DNA).

9. The composition according to claim 8, wherein the nucleotide at the 5′ side and/or the nucleotide at the 3′ side of the orphan nucleotide is DNA.

10. The composition according to any one of claims 1 to 9, wherein the Editing AON comprises at least one phosphonoacetate internucleoside linkage, at least one methylphosphonate internucleoside linkage, and/or at least one nucleotide comprising an unlocked nucleic acid (UNA) ribose modification.

11. The composition according to any one of claims 1 to 10, wherein the Editing AON is 19, 20, or 21 nucleotides in length, wherein the orphan nucleotide is the 6^{th}, 7^{th} or 8^{th} nucleotide from the 5′ end, and wherein the Helper AON is 17, 18, or 19 nucleotides in length.

12. The composition according to any one of claims 1 to 11, further comprising a pharmaceutically acceptable carrier.

13. The composition according to any of claims 1 to 12 for use in therapy, preferably for use in the treatment or prevention of a genetic disorder, preferably selected from the group consisting of: Cystic fibrosis, Hurler Syndrome, alpha-1-antitrypsin (A1AT) deficiency, Parkinson's disease, Alzheimer's disease, albinism, Amyotrophic lateral sclerosis, Asthma, β-thalassemia, CADASIL, Charcot-Marie-Tooth disease, Chronic Obstructive Pulmonary Disease (COPD), Distal Spinal Muscular Atrophy (DSMA), Duchenne/Becker muscular dystrophy, (Dystrophic) Epidermolysis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber Congenital Amaurosis (such as LCA10), Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-esol related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anemia, Spinal Muscular Atrophy, Stargardt disease, Tay-Sachs Disease, Usher syndrome (such as Usher syndrome type I, type II, and type III), X-linked immunodeficiency, Sturge-Weber Syndrome, and cancer.

14. A method for the deamination of at least one target adenosine in a target RNA in a cell, the method comprising the steps of:
(i) providing the cell with a set of AONs comprising a Helper AON and an Editing AON as defined in any one of claims 1 to 11;
(ii) allowing annealing of the AONs to the target RNA to form a double stranded nucleic acid molecule;
(iii) allowing an ADAR enzyme endogenously present in said cell to complex with the double stranded nucleic acid molecule and to deaminate the target adenosine in the target RNA to an inosine; and
(iv) optionally identifying the presence of the deaminated nucleotide in the target RNA.

15. The method of claim 14, wherein step (iv) comprises:
a) sequencing a region of the target RNA, wherein the region comprises the position of the target adenosine;
b) assessing the presence of a functional, elongated, full length and/or wild type protein when the target adenosine is in a UGA or UAG stop codon, which is edited to a UGG codon through the deamination;
c) assessing, when the target RNA is pre-mRNA, whether splicing of the pre-mRNA was altered by the deamination; or
d) using a functional read-out, wherein the target RNA after the deamination encodes a functional, full length, elongated and/or wild type protein.

16. A method for the deamination of at least one target adenosine, present in a target RNA, the method comprising the steps of:
(i) providing a set of two AONs as characterized in any one of claims 1 to 11;
(ii) allowing annealing of the AONs to the target RNA to form a double stranded nucleic acid molecule;
(iii) allowing an ADAR enzyme to complex with the double stranded nucleic acid molecule and to deaminate the target adenosine in the target RNA to an inosine; and
(iv) identifying the presence of the deaminated adenosine in the target RNA.
